SEQUENCE LISTING (12 sequences)

SEQ ID NO: 1 - RNA, 22 nt, Unknown
  Description of Unknown miRNA-21 sequence
uagcuuaucagacugauguuga

SEQ ID NO: 2 - RNA, 22 nt, Unknown
  Description of Unknown miRNA-132 sequence
uaacagucuacagccauggucg

SEQ ID NO: 3 - RNA, 21 nt, Unknown
  Description of Unknown miRNA-9 sequence
uaaagcuagauaaccgaaagu

SEQ ID NO: 4 - RNA, 21 nt, Unknown
  Description of Unknown miRNA-27a sequence
uucacaguggcuaaguuccgc

SEQ ID NO: 5 - RNA, 23 nt, Unknown
  Description of Unknown miRNA-221 sequence
agcuacauugucugcuggguuuc

SEQ ID NO: 6 - RNA, 22 nt, Unknown
  Description of Unknown miRNA-200a-3p sequence
uaacacugucugguaacgaugu

SEQ ID NO: 7 - RNA, 22 nt, Unknown
  Description of Unknown miRNA-361 sequence
uuaucagaaucuccagggguac

SEQ ID NO: 8 - RNA, 22 nt, Unknown
  Description of Unknown miRNA-274 sequence
gaaguuguucgugguggauucg

SEQ ID NO: 9 - RNA, 21 nt, Unknown
  Description of Unknown miR-873a-5p sequence
gcaggaacuugugagucuccu

SEQ ID NO: 10 - protein, 366 aa, Unknown
  Description of Unknown LAMP-2 sequence
MVCFRLFPVPGSGLVLVCLVLGAVRSYALELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAQ

SEQ ID NO: 11 - protein, 29 aa, Rabies lyssavirus
YTIWMPENPRPGTPCDIFTNSRGKRASNG

SEQ ID NO: 12 - DNA, 7333 bp, Artificial Sequence
  Description of Artificial Sequence Synthetic polynucleotide
ccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcactctcctgtggagccagactcctcctcgggaaccggaaaggcgggccagcagcctgcaagaaaaagattgaattttggtcagactggagacgcagactcagtcccagaccctcaaccaatcggagaacctcccgcagccccctcaggtgtgggatctcttacaatggctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacgacaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagcaaggcaacacacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctccgcctcagatcctgatcaagaacacgcctgtacctgcggatcctccgaccaccttcaaccagtcaaagctgaactctttcatcacccagtattctactggccaagtcagcgtggagatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaattgcctgttaatcaataaaccggttgattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggcggccgctcgactagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgatcgaggtcgacggtatcgggggagctcgcagggtctccattttgaagcgggaggtttgaacgcgcag

(19) United States
(12) Patent Application Publication
(10) Pub. No.: US 2023/0075630 A1
(43) Pub. Date: Mar. 9, 2023

(54) EXTRACELLULAR VESICLE-BASED AGENTS AND METHODS FOR THE TREATMENT OF NEUROPATHIC DISORDERS

(71) Applicants: The Johns Hopkins University, Baltimore, MD (US); University of Washington, Seattle, WA (US)

(72) Inventors: Deok-Ho Kim, Baltimore, MD (US); Changho Chun, Seattle, WA (US); Alexander Smith, Seattle, WA (US); Minjae Do, Seoul (KR)

(21) Appl. No.: 17/769,705

(22) PCT Filed: Oct. 16, 2020
(86) PCT No.: PCT/US2020/056092

§ 371 Date: Apr. 15, 2022

Related U.S. Application Data
(60) Provisional application No. 62/916,208, filed on Oct. 16, 2019.

(51) Int. Cl. A61K 35/30; A61K 31/7105; A61K 35/76; A61K 38/18; A61K 38/17; A61K 47/69; C12N 5/079; C12N 5/0793

(52) U.S. Cl. CPC A61K 35/30; A61K 31/7105; A61K 35/76; A61K 38/185; A61K 38/1709; A61K 47/6911; C12N 5/0622; C12N 5/0619; A61K 48/00

(57) ABSTRACT

Provided herein, inter alia, are compositions and methods comprising glial-derived extracellular vesicles for the prevention and treatment of neuropathies. In aspects, the glial-derived extracellular vesicles may include one or more of the following miRNA, an adeno-associated virus (AAV), siRNA, vRNA, mRNA, lncRNA, DNA, tetraspanins, amino acids, metabolites, signaling proteins, chaperones, cytoskeletal proteins, enzymes, or combinations thereof.

### RELATED APPLICATIONS

This application claims the benefit of priority under 35 U.S.C. § 119(e) to U.S. Provisional Application No. 62/916,208 filed Oct. 16, 2019, the entire contents of which is incorporated herein by reference in its entirety.

### STATEMENT AS TO FEDERALLY SPONSORED RESEARCH

This invention was made with government support under Grant Number: R01 NS094388 awarded by the National Institute of Neurological Disorders and Stroke. The Government has certain rights in the invention.

### BACKGROUND

New compositions and methods for treating neuropathies are provided.

### BRIEF SUMMARY

Provided herein, inter alia, are compositions and methods comprising glial-derived extracellular vesicles for the prevention and treatment of neuropathies, e.g., peripheral nerve system (PNS) neuropathies and central nervous system neuropathies (CNS). In aspects, the glial-derived extracellular vesicles may include one or more of the following miRNA, an adeno-associated virus (AAV), siRNA, vRNA, mRNA, lncRNA, DNA, tetraspanins, amino acids, metabolites, signaling proteins, chaperones, cytoskeletal proteins, enzymes, or combinations thereof.

In embodiments, the glial-derived extracellular vesicles include miRNA-21, miRNA-132, miRNA-9, miRNA-27a, miRNA-221, miRNA-200a-3p, miRNA-361, miRNA-274, miR-873a-5p, AAV1, AAV2, AAV4, AAV5, AAV6, AAV7, AAV8, AAV9 AAVDJ8, AAVrh10, glial derived neurotrophic factor, brain derived neurotrophic factor, Ciliary neurotrophic factor, glial fibrillary acidic protein, or combinations thereof. Exemplary sequences include:

miRNA-21
(SEQ ID NO: 1)
UAGCUUAUCAGACUGAUGUUGA,
miRNA-132:
(SEQ ID NO: 2)
UAACAGUCUACAGCCAUGGUCG,
miRNA-9:
(SEQ ID NO: 3)
UAAAGCUAGAUAACCGAAAGU,
miRNA-27a:
(SEQ ID NO: 4)
UUCACAGUGGCUAAGUUCCGC,
miRNA-221:
(SEQ ID NO: 5)
AGCUACAUUGUCUGCUGGGUUUC,
miRNA-200a-3p:
(SEQ ID NO: 6)
UAACACUGUCUGGUAACGAUGU,
miRNA-361
(SEQ ID NO: 7)
UUAUCAGAAUCUCCAGGGGUAC,
miRNA-274:
(SEQ ID NO: 8)
GAAGUUGUUCGUGGUGGAUUCG,
miR-873a-5p:
(SEQ ID ON: 9)
GCAGGAACUUGUGAGUCUCCU.

For example, the glial-derived extracellular vesicles include miRNA-21, miRNA-132, miRNA-9, miRNA-27a, miRNA-221, miRNA-200a-3p, miRNA-361, miRNA-274, or miR-873a-5p. In other examples, the glial-derived extracellular vesicles include AAV1, AAV2, AAV4, AAV5, AAV6, AAV7, AAV8, AAV9 AAVDJ8, AAVrh10, or combinations thereof. Additional exemplary cargos may be found at Goetzl E J, Mustapic M, Kapogiannis D, et al. Cargo proteins of plasma astrocyte-derived exosomes in Alzheimer's disease. FASEB J. 2016; 30(11):3853-3859, incorporated herein by reference in its entirety.

In embodiments, the glial-derived extracellular vesicles are derived from astrocytes, Schwann cells, oligodendrocytes, ependymal cells, microglia, or satellite cells in 2D or 3D cultures. For example, spheroid, stem cell-derived organoid, or tissue engineered 3D constructs made of astrocytes and/or Schwann cells and/or oligodendrocytes. It is contemplated that this 3D culture (including spheroid, stem cell-derived organoid, or tissue engineered 3D constructs made of astrocytes and/or Schwann cells and/or oligodendrocytes) promotes increased throughput and/or yield of extracellular vesicle (exosome) production.

3D tissue spheroids made of glial cells (e.g. astrocytes or Swanne cells) were generated by using AggreWell 400 6-well microwell culture plates (STEMCELL Technologies, USA). Before seeding cells in microwell culture plates, 0.5 mL anti-adherence rinsing solution (STEMCELL Technologies, USA) was added into each well, and a 2-minute 2000 g centrifugation followed by a 30-minute incubation at room temperature was performed to prevent cell adhesion onto the microwells. Next, a 2.5 million single-cell suspension in 2 mL was seeded in each well, a 5-minute 200 g centrifugation was performed to cluster the cells in microwells, and the cells were incubated in a CO₂ incubator at 37° C. overnight for cells to aggregate and form spheroids. See, e.g., G. Razian, Y. Yu, M. Ungrin, Production of large numbers of size-controlled tumor spheroids using microwell plates, J Vis Exp (81) (2013) e50665, incorporated herein by reference in its entirety.

In examples, the cells are mammalian (e.g., human) primary isolated cells or mammalian (e.g., human) stem cells. The mammalian stem cells, for example include induced pluripotent stem cells (iPSC), embryonic stem cells (ESCs), or mesenchymal stem cells (MSCs).

In embodiments, the glial-derived extracellular vesicles include an adeno-associated virus (AAV) vector. For example, the AAV includes AAV9, AAVDJ8, AAVrh10, AAV6, AAV5, AAV1, or AAV2. Sequence information is publically available, for example, at Cell Biolabs, Inc. An exemplary sequence of AADJ8 is shown below (SEQ ID NO: 12).

6-1871: AAV-2 Rep gene

1888-4101: AAV-DJ/8 Cap gene

5606-6466: Ampicillin Resistance (Bla) Gene (Complement)

CCGCCATGCCGGGGTTTTACGAGATTGTGATTAAGGTCCCCAGCGACCTTGACGAGCATCTGCC
CGGCATTTCTGACAGCTTTGTGAACTGGGTGGCCGAGAAGGAATGGGAGTTGCCGCCAGATTCT
GACATGGATCTGAATCTGATTGAGCAGGCACCCCTGACCGTGGCCGAGAAGCTGCAGCGCGACT
TTCTGACGGAATGGCGCCGTGTGAGTAAGGCCCCGGAGGCCCTTTTCTTTGTGCAATTTGAGAA
GGGAGAGAGCTACTTCCACATGCACGTGCTCGTGGAAACCACCGGGGTGAAATCCATGGTTTTG
GGACGTTTCCTGAGTCAGATTCGCGAAAAACTGATTCAGAGAATTTACCGCGGGATCGAGCCGA
CTTTGCCAAACTGGTTCGCGGTCACAAAGACCAGAAATGGCGCCGGAGGCGGGAACAAGGTGGT
GGATGAGTGCTACATCCCCAATTACTTGCTCCCCAAAACCCAGCCTGAGCTCCAGTGGGCGTGG
ACTAATATGGAACAGTATTTAAGCGCCTGTTTGAATCTCACGGAGCGTAAACGGTTGGTGGCGC
AGCATCTGACGCACGTGTCGCAGACGCAGGAGCAGAACAAAGAGAATCAGAATCCCAATTCTGA
TGCGCCGGTGATCAGATCAAAAACTTCAGCCAGGTACATGGAGCTGGTCGGGTGGCTCGTGGAC
AAGGGGATTACCTCGGAGAAGCAGTGGATCCAGGAGGACCAGGCCTCATACATCTCCTTCAATG
CGGCCTCCAACTCGCGGTCCCAAATCAAGGCTGCCTTGGACAATGCGGGAAAGATTATGAGCCT
GACTAAAACCGCCCCCGACTACCTGGTGGGCCAGCAGCCCGTGGAGGACATTTCCAGCAATCGG
ATTTATAAAATTTTGGAACTAAACGGGTACGATCCCCAATATGCGGCTTCCGTCTTTCTGGGAT
GGGCCACGAAAAAGTTCGGCAAGAGGAACACCATCTGGCTGTTTGGGCCTGCAACTACCGGGAA
GACCAACATCGCGGAGGCCATAGCCCACACTGTGCCCTTCTACGGGTGCGTAAACTGGACCAAT
GAGAACTTTCCCTTCAACGACTGTGTCGACAAGATGGTGATCTGGTGGGAGGAGGGGAAGATGA
CCGCCAAGGTCGTGGAGTCGGCCAAAGCCATTCTCGGAGGAAGCAAGGTGCGCGTGGACCAGAA
ATGCAAGTCCTCGGCCCAGATAGACCCGACTCCCGTGATCGTCACCTCCAACACCAACATGTGC
GCCGTGATTGACGGGAACTCAACGACCTTCGAACACCAGCAGCCGTTGCAAGACCGGATGTTCA
AATTTGAACTCACCCGCCGTCTGGATCATGACTTTGGGAAGGTCACCAAGCAGGAAGTCAAAGA
CTTTTTCCGGTGGGCAAAGGATCACGTGGTTGAGGTGGAGCATGAATTCTACGTCAAAAAGGGT
GGAGCCAAGAAAAGACCCGCCCCCAGTGACGCAGATATAAGTGAGCCCAAACGGGTGCGCGAGT
CAGTTGCGCAGCCATCGACGTCAGACGCGGAAGCTTCGATCAACTACGCAGACAGGTACCAAAA
CAAATGTTCTCGTCACGTGGGCATGAATCTGATGCTGTTTCCCTGCAGACAATGCGAGAGAATG
AATCAGAATTCAAATATCTGCTTCACTCACGGACAGAAAGACTGTTTAGAGTGCTTTCCCGTGT
CAGAATCTCAACCCGTTTCTGTCGTCAAAAAGGCGTATCAGAAACTGTGCTACATTCATCATAT
CATGGGAAAGGTGCCAGACGCTTGCACTGCCTGCGATCTGGTCAATGTGGATTTGGATGACTGC
ATCTTTGAACAATAAATGATTTAAATCAGGTATGGCTGCCGATGGTTATCTTCCAGATTGGCTC
GAGGACACTCTCTCTGAAGGAATAAGACAGTGGTGGAAGCTCAAACCTGGCCCACCACCACCAA
AGCCCGCAGAGCGGCATAAGGACGACAGCAGGGGTCTTGTGCTTCCTGGGTACAAGTACCTCGG
ACCCTTCAACGGACTCGACAAGGGAGAGCCGGTCAACGAGGCAGACGCCGCGGCCCTCGAGCAC
GACAAAGCCTACGACCGGCAGCTCGACAGCGGAGACAACCCGTACCTCAAGTACAACCACGCCG
ACGCCGAGTTCCAGGAGCGGCTCAAAGAAGATACGTCTTTTGGGGGCAACCTCGGGCGAGCAGT
CTTCCAGGCCAAAAAGAGGCTTCTTGAACCTCTTGGTCTGGTTGAGGAAGCGGCTAAGACGGCT
CCTGGAAAGAAGAGGCCTGTAGAGCACTCTCCTGTGGAGCCAGACTCCTCCTCGGGAACCGGAA
AGGCGGGCCAGCAGCCTGCAAGAAAAAGATTGAATTTTGGTCAGACTGGAGACGCAGACTCAGT
CCCAGACCCTCAACCAATCGGAGAACCTCCCGCAGCCCCCTCAGGTGTGGGATCTCTTACAATG
GCTGCAGGCGGTGGCGCACCAATGGCAGACAATAACGAGGGCGCCGACGGAGTGGGTAATTCCT
CGGGAAATTGGCATTGCGATTCCACATGGATGGGCGACAGAGTCATCACCACCAGCACCCGAAC
CTGGGCCCTGCCCACCTACAACAACCACCTCTACAAGCAAATCTCCAACAGCACATCTGGAGGA
TCTTCAAATGACAACGCCTACTTCGGCTACAGCACCCCCTGGGGGTATTTTGACTTTAACAGAT
TCCACTGCCACTTTTCACCACGTGACTGGCAGCGACTCATCAACAACAACTGGGGATTCCGGCC
CAAGAGACTCAGCTTCAAGCTCTTCAACATCCAGGTCAAGGAGGTCACGCAGAATGAAGGCACC
AAGACCATCGCCAATAACCTCACCAGCACCATCCAGGTGTTTACGGACTCGGAGTACCAGCTGC
CGTACGTTCTCGGCTCTGCCCACCAGGGCTGCCTGCCTCCGTTCCCGGCGGACGTGTTCATGAT
TCCCCAGTACGGCTACCTAACACTCAACAACGGTAGTCAGGCCGTGGGACGCTCCTCCTTCTAC
TGCCTGGAATACTTTCCTTCGCAGATGCTGAGAACCGGCAACAACTTCCAGTTTACTTACACCT
TCGAGGACGTGCCTTTCCACAGCAGCTACGCCCACAGCCAGAGCTTGGACCGGCTGATGAATCC
TCTGATTGACCAGTACCTGTACTACTTGTCTCGGACTCAAACAACAGGAGGCACGACAAATACG
CAGACTCTGGGCTTCAGCCAAGGTGGGCCTAATACAATGGCCAATCAGGCAAAGAACTGGCTGC
CAGGACCCTGTTACCGCCAGCAGCGAGTATCAAAGACATCTGCGGATAACAACAACAGTGAATA
CTCGTGGACTGGAGCTACCAAGTACCACCTCAATGGCAGAGACTCTCTGGTGAATCCGGGCCCG
GCCATGGCAAGCCACAAGGACGATGAAGAAAAGTTTTTTCCTCAGAGCGGGGTTCTCATCTTTG
GGAAGCAAGGCTCAGAGAAAACAAATGTGGACATTGAAAAGGTCATGATTACAGACGAAGAGGA
AATCAGGACAACCAATCCCGTGGCTACGGAGCAGTATGGTTCTGTATCTACCAACCTCCAGCAA
GGCAACACACAAGCAGCTACCGCAGATGTCAACACACAAGGCGTTCTTCCAGGCATGGTCTGGC
AGGACAGAGATGTGTACCTTCAGGGGCCCATCTGGGCAAAGATTCCACACACGGACGGACATTT
TCACCCCTCTCCCCTCATGGGTGGATTCGGACTTAAACACCCTCCGCCTCAGATCCTGATCAAG
AACACGCCTGTACCTGCGGATCCTCCGACCACCTTCAACCAGTCAAAGCTGAACTCTTTCATCA
CCCAGTATTCTACTGGCCAAGTCAGCGTGGAGATCGAGTGGGAGCTGCAGAAGGAAAACAGCAA
GCGCTGGAACCCCGAGATCCAGTACACCTCCAACTACTACAAATCTACAAGTGTGGACTTTGCT
GTTAATACAGAAGGCGTGTACTCTGAACCCCGCCCCATTGGCACCCGTTACCTCACCCGTAATC
TGTAATTGCCTGTTAATCAATAAACCGGTTGATTCGTTTCAGTTGAACTTTGGTCTCTGCGAAG
GGCGAATTCGTTTAAACCTGCAGGACTAGAGGTCCTGTATTAGAGGTCACGTGAGTGTTTTGCG
ACATTTTGCGACACCATGTGGTCACGCTGGGTATTTAAGCCCGAGTGAGCACGCAGGGTCTCCA
TTTTGAAGCGGGAGGTTTGAACGCGCAGCCGCCAAGCCGAATTCTGCAGATATCCATCACACTG
GCGGCCGCTCGACTAGAGCGGCCGCCACCGCGGTGGAGCTCCAGCTTTTGTTCCCTTTAGTGAG
GGTTAATTGCGCGCTTGGCGTAATCATGGTCATAGCTGTTTCCTGTGTGAAATTGTTATCCGCT
CACAATTCCACACAACATACGAGCCGGAAGCATAAAGTGTAAAGCCTGGGGTGCCTAATGAGTG
AGCTAACTCACATTAATTGCGTTGCGCTCACTGCCCGCTTTCCAGTCGGGAAACCTGTCGTGCC
AGCTGCATTAATGAATCGGCCAACGCGCGGGGAGAGGCGGTTTGCGTATTGGGCGCTCTTCCGC
TTCCTCGCTCACTGACTCGCTGCGCTCGGTCGTTCGGCTGCGGCGAGCGGTATCAGCTCACTCA
AAGGCGGTAATACGGTTATCCACAGAATCAGGGGATAACGCAGGAAAGAACATGTGAGCAAAAG
GCCAGCAAAAGGCCAGGAACCGTAAAAAGGCCGCGTTGCTGGCGTTTTTCCATAGGCTCCGCCC
CCCTGACGAGCATCACAAAAATCGACGCTCAAGTCAGAGGTGGCGAAACCCGACAGGACTATAA
AGATACCAGGCGTTTCCCCCTGGAAGCTCCCTCGTGCGCTCTCCTGTTCCGACCCTGCCGCTTA
CCGGATACCTGTCCGCCTTTCTCCCTTCGGGAAGCGTGGCGCTTTCTCATAGCTCACGCTGTAG
GTATCTCAGTTCGGTGTAGGTCGTTCGCTCCAAGCTGGGCTGTGTGCACGAACCCCCCGTTCAG
CCCGACCGCTGCGCCTTATCCGGTAACTATCGTCTTGAGTCCAACCCGGTAAGACACGACTTAT
CGCCACTGGCAGCAGCCACTGGTAACAGGATTAGCAGAGCGAGGTATGTAGGCGGTGCTACAGA
GTTCTTGAAGTGGTGGCCTAACTACGGCTACACTAGAAGAACAGTATTTGGTATCTGCGCTCTG
CTGAAGCCAGTTACCTTCGGAAAAAGAGTTGGTAGCTCTTGATCCGGCAAACAAACCACCGCTG
GTAGCGGTGGTTTTTTTGTTTGCAAGCAGCAGATTACGCGCAGAAAAAAAGGATCTCAAGAAGA
TCCTTTGATCTTTTCTACGGGGTCTGACGCTCAGTGGAACGAAAACTCACGTTAAGGGATTTTG
GTCATGAGATTATCAAAAAGGATCTTCACCTAGATCCTTTTAAATTAAAAATGAAGTTTTAAAT
CAATCTAAAGTATATATGAGTAAACTTGGTCTGACAGTTACCAATGCTTAATCAGTGAGGCACC
TATCTCAGCGATCTGTCTATTTCGTTCATCCATAGTTGCCTGACTCCCCGTCGTGTAGATAACT
ACGATACGGGAGGGCTTACCATCTGGCCCCAGTGCTGCAATGATACCGCGAGACCCACGCTCAC
CGGCTCCAGATTTATCAGCAATAAACCAGCCAGCCGGAAGGGCCGAGCGCAGAAGTGGTCCTGC
AACTTTATCCGCCTCCATCCAGTCTATTAATTGTTGCCGGGAAGCTAGAGTAAGTAGTTCGCCA
GTTAATAGTTTGCGCAACGTTGTTGCCATTGCTACAGGCATCGTGGTGTCACGCTCGTCGTTTG
GTATGGCTTCATTCAGCTCCGGTTCCCAACGATCAAGGCGAGTTACATGATCCCCCATGTTGTG
CAAAAAAGCGGTTAGCTCCTTCGGTCCTCCGATCGTTGTCAGAAGTAAGTTGGCCGCAGTGTTA
TCACTCATGGTTATGGCAGCACTGCATAATTCTCTTACTGTCATGCCATCCGTAAGATGCTTTT
CTGTGACTGGTGAGTACTCAACCAAGTCATTCTGAGAATAGTGTATGCGGCGACCGAGTTGCTC
TTGCCCGGCGTCAATACGGGATAATACCGCGCCACATAGCAGAACTTTAAAAGTGCTCATCATT
GGAAAACGTTCTTCGGGGCGAAAACTCTCAAGGATCTTACCGCTGTTGAGATCCAGTTCGATGT
AACCCACTCGTGCACCCAACTGATCTTCAGCATCTTTTACTTTCACCAGCGTTTCTGGGTGAGC
AAAAACAGGAAGGCAAAATGCCGCAAAAAAGGGAATAAGGGCGACACGGAAATGTTGAATACTC
ATACTCTTCCTTTTTCAATATTATTGAAGCATTTATCAGGGTTATTGTCTCATGAGCGGATACA
TATTTGAATGTATTTAGAAAAATAAACAAATAGGGGTTCCGCGCACATTTCCCCGAAAAGTGCC
ACCTAAATTGTAAGCGTTAATATTTTGTTAAAATTCGCGTTAAATTTTTGTTAAATCAGCTCAT
TTTTTAACCAATAGGCCGAAATCGGCAAAATCCCTTATAAATCAAAAGAATAGACCGAGATAGG
GTTGAGTGTTGTTCCAGTTTGGAACAAGAGTCCACTATTAAAGAACGTGGACTCCAACGTCAAA
GGGCGAAAAACCGTCTATCAGGGCGATGGCCCACTACGTGAACCATCACCCTAATCAAGTTTTT
TGGGGTCGAGGTGCCGTAAAGCACTAAATCGGAACCCTAAAGGGAGCCCCCGATTTAGAGCTTG
ACGGGGAAAGCCGGCGAACGTGGCGAGAAAGGAAGGGAAGAAAGCGAAAGGAGCGGGCGCTAGG
GCGCTGGCAAGTGTAGCGGTCACGCTGCGCGTAACCACCACACCCGCCGCGCTTAATGCGCCGC
TACAGGGCGCGTCCCATTCGCCATTCAGGCTGCGCAACTGTTGGGAAGGGCGATCGGTGCGGGC
CTCTTCGCTATTACGCCAGCTGGCGAAAGGGGGATGTGCTGCAAGGCGATTAAGTTGGGTAACG
CCAGGGTTTTCCCAGTCACGACGTTGTAAAACGACGGCCAGTGAGCGCGCGTAATACGACTCAC
TATAGGGCGAATTGGGTACCGGGCCCCCCCTCGATCGAGGTCGACGGTATCGGGGGAGCTCGCA
GGGTCTCCATTTTGAAGCGGGAGGTTTGAACGCGCAG

In embodiments, the glial-derived extracellular vesicle includes a genetically modified protein or fragment thereof for expressing the protein or fragment thereof on the surface of the extracellular vesicle. For example, the genetically modified protein comprises Lamp-1, Lamp-2, tetraspanins, CD2, CD3, CD9, CD13, CD18, CD36, CD37, CD40, CD40L, CD41a, CD44, CD45, CD53, CD63, CD81, CD82, CD86, Flotillin, Syntaxin-3, ICAM-1, Integrin alpha4, LiCAM, LFA-1, Mac-1, Vti-1A and B, CXCR4, FcR, GluR2/3, HLA-DM, Immunoglobulins, MHC-1, MHC-2, or TCR beta. Exemplary sequences include: Lamp-2:

(SEQ ID NO: 10)
mvcfrlfpvpgsglvlvclvlgavrsyalelnltdsenatclyakwqmn
ftvryettnktyktvtisdhgtvtyngsicgddqngpkiavqfgpgfsw
ianftkaastysidsvsfsyntgdnttfpdaedkgiltvdellairipl
ndlfrcnslstlekndvvqhywdvlvqafvqngtvstneflcdkdktst
vaptihttvpsptttptpkekpeagtysvnngndtcllatmglqlnitq
dkvasvininpntthstgscrshtallrlnsstikyldfvfavknenrf
ylkevnismylvngsvfsiannnlsywdaplgssymcnkeqtvsvsgaf
qintfdlrvqpfnvtqgkystaq.

In other examples, the genetically modified protein comprises at least one of a rabies virus glycoprotein (RVG), a tetanus toxin fragment C, or an RGD peptide. RVG:

(SEQ ID NO: 11)
YTIWMPENPRPGTPCDIFTNSRGKRASNG.

In embodiments, the glial-derived extracellular vesicle has a diameter from about 10 nm to about 1000 nm. Alternatively, the glial-derived extracellular vesicle has a diameter from about 50 nm to about 200 nm. In other examples, the glial-derived exosome (e.g., a glial-derived exosome) is characterized as having a diameter from about 10 nm to about 5000 nm, from about 10 nm to about 1000 nm, e.g., a diameter from about 10 nm to about 300 nm, from about, from about 30 nm to about 150 nm, or from about 30 nm to about 100 nm.

In aspects, also provided herein is a method for preparing the glial-derived extracellular vesicle, including culturing cells in a medium, wherein the cells release the extracellular vesicle by secretion into the medium, collecting the supernatant of medium, fractionating the supernatant comprising the extracellular vesicle, and isolating the extracellular vesicle.

In embodiments, the method further comprises fractionation by ultracentrifugation, ultrafiltration, tangential flow filtration (TFF), or a combination thereof.

For example, methods can be used to increase exosome production and exosome yield. For example, methods of maximizing production efficiency using acoustic stimulus or electric stimulus (e.g., during EV biogenesis) are contemplated. See, e.g., Commun Biol. 2020 Oct. 5; 3(1):553, and Nat Biomed Eng. 2020 January; 4(1):69-83, incorporated herein by reference in their entireties.

In embodiments, the method provides that the cells are derived from the human neuronal ectoderm, neuronal supporting cells in the peripheral nervous system, or neuronal supporting cells in the central nervous system. For example, the cells include astrocytes, Schwann cells, oligodendrocytes, ependymal cells, microglia, or satellite cells in 2D or 3D cultures.

In embodiments, the method further may include one or more vacuum mediated centrifugation steps, wherein the vacuum mediated centrifugation removes live cells, dead cell debris, larger cellular debris, or combinations thereof.

In aspects, provided herein is a method of treating a neuropathy in a patient comprising administering to the patient a composition including the glial-derived extracellular vesicles described herein.

For example, the neuropathy includes traumatic peripheral nerve injury, chemotherapy induced peripheral neuropathy, traumatic brain injury, stroke, Charcot-Marie-Tooth disease (CMT), Amyotrophic Lateral Sclerosis (ALS), Parkinson's disease, Alzheimer's disease, frontotemporal dementia, Huntington's disease, Multiple Sclerosis, Congenital Myasthenia, Apraxia, Hypertonia, myasthenia gravis, or spinal muscular atrophy.

In embodiments, the method for treating the neuropathy includes that the composition is administered locally or systemically. In other examples, the composition is administered via intravenous, ultrasonic, intrathecal, or subcutaneous administration.

In embodiments, the patient is a mammal, for example, a human.

In aspects, provided herein is a method for preventing or treating neuronal apoptosis, neuronal senescence, neuritic outgrowth, synapse function or electrophysiological function, comprising administering the composition including the glial-derived extracellular vesicle.

In embodiments, the neuronal apoptosis, neuronal senescence or electrophysiological function comprises cell death, neurite degeneration, abnormal resting membrane potential, abnormal repetitive firing behavior, repolarizing velocity, and action potential firing frequency.

In other examples, the neuronal apoptosis is decreased by about 5%, 10%, 20%, 30%, 40%, 50%, or 100%, compared to a patient not administered the composition. Moreover, the electrophysiological function is increased by about 5%, 10%, 20%, 30%, 40%, 50%, 100%, 150%, or 200% compared to a patient not administered the composition.

In other embodiments, the neuritic outgrowth is increased by about 5%, 10%, 20%, 30%, 40%, 50%, 100%, 150%, or 200% compared to a patient not administered the composition. In embodiments, the synapse formation/function is increased by about 5%, 10%, 20%, 30%, 40%, 50%, 100%, 150%, or 200% compared to a patient not administered the composition.

In embodiments, the glial-derived exosomes can be targeted to a desired cell type or tissue. This targeting can be achieved by expressing on the surface of the exosome a targeting moiety which binds to a cell surface moiety expressed on the surface of the cell to be targeted. Typically the targeting moiety is a peptide within the disclosed exosome targeted fusion protein. However, it can also be independently expressed as a fusion protein with an exosomal transmembrane moiety.

In other aspects, provided herein is a composition including a glial-derived extracellular vesicle (or a glial-derived exosome), and where the extracellular vesicle has one or more gene editing tools. For example the gene editing tools include a gene editing protein, an RNA molecule and/or a ribonucleoprotein.

In embodiments, the gene editing protein is a zinc finger nuclease (ZFN), a transcription activator-like effector nuclease (TALEN), a MegaTAL, a Cas protein, a Cre recombinase, a Hin recombinase, or a Flp recombinase. In other examples, the RNP includes a Cas protein.

Kits that include one or more reagents for preparing the glial-derived extracellular vesicle (or an exosome) wherein the exosome is also within the invention.

In a further aspect, the present extracellular vesicles may be used in in vitro and ex vivo systems. For instance, stem cells or progenitor cells may be treated or incubated with the present extracellular vesicles to promote or induce functionality and/or maturation. In such methods, for example, a population of cells, for instance stem cells or progenitor cells may be obtained; and the present extracellular vesicles administered to the population of cells in vitro, suitably to generate functionality or maturation of the treated cells. In some embodiments, the present extracellular vesicles may be administered (e.g. incubated) for at least 1, 2, 3, 6, 12, 18 or 24 hours to the population of cells in vitro. In an additional aspect, a population of cells is provided that have been treated in vitro with the present extracellular vesicles as disclosed herein.

Other aspects of the invention are disclosed infra.

### DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C are data showing the differentiation of cortical neurons from NGN2 transgene transduced human iPSCs. FIG. 1A is a schematic showing the differentiation protocol highlighting the use of doxycyclin to initiate NGN2 transcription. FIG. 1B are images showing the morphological changes observed between initial stages of differentiation and the later stages of culture. FIG. 1C are images showing human iPSC-derived cortical neurons immunostained using antibodies against CUX-1 (neuronal marker in cerebral cortex layers 2 and 3) and MAP2 (pan-neuronal marker) with DAPI-stained nuclei. Inset shows clear expression of CUX-1 in the cell cytoplasm. Scale bar of inset image: 20 μm.

FIGS. 2A-2J are data showing EV characterization and internalization into cultured cortical neurons. FIG. 2A is a bright field image of human primary astrocytes. FIG. 2B are images showing astrocyte culture immunostained using antibodies against GFAP and S100B; glial cell markers primarily expressed in astrocytes. FIG. 2C is a bar graph showing the size distribution of collected EVs. The average particle size calculated from these experiments was 154.6±4.36 nm. FIG. 2D is a graph showing the size distribution of collected EVs with various cell incubation times before EV collection. FIG. 2E is a bar graph showing the number of EVs collected after different lengths of time in culture. Incubation times on the x axis indicate the time elapsed following EV-free medium change at day 4. Error bars are standard error mean (SEM). FIG. 2F are images showing transmission electron microscope (TEM) images of EVs collected from conditioned medium after 84 hours of astrocyte incubation in exogenous EV-free condition. FIG. 2G is a bar graph showing the mode size of EVs collected from cultures at various cell incubation times. The mean value is 124 nm. FIG. 2H are images showing representative cortical neurons double immunostained with antibodies against CD81 and F-actin to show internalization of EVs. The ratio of EV to cell in the treated example are indicated in the legend. FIG. 2I is a bar graph showing uptake efficiency of EVs at different ratio of neurons to EVs calculated by quantifying the number of cells with fluorescently labeled EVs. FIG. 2J is a graph showing the relationship between the CD81 fluorescent intensity in EV-treated cells and the ratio of EV to cell used to treat the cultures. The fluorescence intensity is expressed as relative values normalized to an untreated sample. In all presented data, **p<0.005 ***p<0.0005 ****p<0.0001, ns not significant. (n=3).

FIGS. 3A and 3B are data showing the analysis of apoptotic neurons in response to treatment with various concentrations of astrocyte-derived EVs. FIG. 3A are images of flow cytometry analysis of neurons at day 6 using Annexin V and propidium iodide (PI). The inset numbers indicate the percentage of cells occupying each quadrant. Q1=early stage apoptotic cells (PI positive), Q2=late stage apoptotic cells (both PI and Annexin V positive), Q3=early stage apoptotic cells (Annexin V positive), Q4=alive cells (PI and Annexin V negative). 10,000 cells per condition were used for this assay. FIG. 3B is a bar graph showing the quantification and comparison of the number of analyzed cells in each condition.

FIGS. 4A-4C are data showing neuronal senescence assay comparing untreated and EV-treated neurons at day 3 post-induction. FIG. 4A are representative bright-field images taken from different time points in culture with and without EV treatment. FIG. 4B are representative bright-field images taken after cells were stained with a β-galactosidase solution that produces a precipitate in the cytoplasm of senescent cells supporting hyperactive lysosomes. Scale bars of inset images: 20 μm. FIG. 4C is a bar graph showing the quantification of the number of β-galactosidase stained cells in treated and untreated cells at day 4 of each culture Error bars are SEM. **p<0.005. (n=3).

FIGS. 5A-5C are data showing neurite outgrowth speed and axon branching comparison between EV untreated and treated neurons. FIG. 5A are images showing a morphological comparison of neurons plated in low density with and without EV treatment at three different time points. FIG. 5B are representative immunostained images of neurons stained for neurofilament expression to label axons at different timepoints. FIG. 5C is a bar graph showing the population of neurons exhibiting different numbers of axon branches in each culture condition. (n=19).

FIGS. 6A-6G are data showing single cell electrophysiology of astrocyte-derived EV-treated and untreated neurons. FIG. 6A is a graph showing representative action potential traces recorded from EV-treated and untreated neurons. FIG. 6B is a graph showing maximum action potential firing rate in each group of neurons measured in response to a 500 ms depolarizing current injection. FIG. 6C is a bar graph showing the number of patched neurons exhibiting different action potential firing patterns, categorized as ‘repetitive’, ‘adaptive’, ‘single’, and ‘none’, as reported previously. ⁴⁹ FIG. 6D is a bar graph showing the action potential duration at 90% of repolarization. Repolarization speed (FIG. 6E) and depolarization speed (FIG. 6F) of neurons relative to the apex of the recorded action potential. FIG. 6G is a bar graph showing the resting membrane potential measured from unstimulated neurons with 0 pA current injection. In all presented data, error bars are SEM, and ***p<0.0003 ****p<0.0001, ns not significant. n=20 (untreated), n=16 (EV-treated) for each data set.

FIG. 7 is a schematic showing an illustration of EV collection process. Each step illustrates the centrifugation speed required to adequately remove non-EV components inside the culture in serial manner.

FIGS. 8A and 8B are bar graphs showing the characterization of astrocyte-derived EVs collected in different batches. FIG. 8A is a bar graph showing the density of EVs in three different batches. FIG. 8B is a bar graph showing the average size of EVs in the same batches. Error bars are SEM.

FIG. 9 is an image showing live cell image of EV-treated neurons at day 6. EVs were fluorescently tagged with ExoGlow dye to visualize the uptake of EVs into the majority cells in live culture.

FIG. 10 is a bar graph showing neurite outgrowth speed for cortical neurons during initial stages of culture. Comparison of the longest neurite (μm) in each analyzed cell at each timepoint in response to different concentrations of EV treatment. Error bars are SEM.

FIGS. 11A and 11B are data from Schwann cell-derived exosomes. FIG. 11A is a microscopic image of human Schwann cells during exosome production (scale bar=100 mm). FIG. 11B is a graph showing the size distribution of produced human Schwann cell derived exosome and its microscopic image (inbox, scale bar=1 mm) The average diameter of the exosome was confirmed to 102.5±105.8 nm by using a NanoSight device.

FIG. 12 is a schematic depicting an extracellular vesicle (or exosome) comprising a targeting peptide expressed on the surface which binds to a cell surface moiety expressed on the surface of the cell to be targeted, e.g., a receptor.

### DETAILED DESCRIPTION

Provided herein are, inter alia, compositions, methods, and kits for treating and preventing neuropathies, including peripheral nervous system (PNS) and central nervous system disorders.

Neurons derived from human induced pluripotent stem cells (hiPSCs) are powerful tools for modeling neural pathophysiology and preclinical efficacy/toxicity screening of novel therapeutic compounds. However, human neurons cultured in vitro typically do not fully recapitulate the physiology of the human nervous system, especially in terms of exhibiting comparable morphological maturation, longevity, and electrochemical signaling ability to adult human neurons.

As described herein, glial cell-derived extracellular vesicles (EVs) were investigated to modulate neuronal survival and electrophysiological function in vitro. Specifically, EVs obtained from human astrocytes promoted enhanced electrophysiological function and anti-apoptotic behavior in hiPSC-derived human cortical neurons. Moreover, EVs isolated from human Schwann cells can be harvested in a similar manner (e.g., in a similar manner as extraction of astrocytes) and have been shown previously to enhance neuron function.

While neurite outgrowth was unaffected by EV treatment, improvements in neuronal function and inhibition of apoptotic signaling in treated cells may hold significant potential for improving preclinical screens utilizing such cells. As such, astrocyte EV-mediated neuronal potentiation in culture holds the potential to expand the utility of hiPSC-derived neurons, particularly in the development of biomimetic human nerve tissue models. In addition, the data highlight the potential for EV-based therapeutics as a potential class of future clinical treatments for tackling inveterate central and peripheral neuropathies.

The cerebral cortex of the mammalian brain is well organized with specific layers supporting distinct neuronal populations.¹⁻³ Within the cortex, neurons not only form synapses with each other, but also with glial cells. These neuron-glia synapses are necessary for ensuring neuron survival, regeneration, differentiation, and proper coordination of motor and sensory information in the brain.³⁻⁹ Astrocytes are the most abundant subtype of glial cells coexisting with neurons in the central nervous system (CNS).¹⁰ They are known to provide structural and trophic support to neurons, mediate synaptogenesis at early stages of neonatal brain development, maintain and eliminate neuronal synapses, and modulate excitability of associated neurons.¹⁰ Animal studies have revealed that astrogenesis, which begins during the later stages of neurogenesis (around E18 in rodents), astrocyte division and expansion coincide with axon and dendrite outgrowth as well as synaptic initiation in neurons.^{10,11} Although numerous studies have revealed a diverse range of astrocyte functionality on neurons, detailed mechanisms of how these nerve cells specifically interact with each other during development and beyond, and how their interactions are altered in diseased tissues are not well understood. In this study, emphasis was on neuronastrocyte paracrine signaling via astrocyte-derived EVs.

Specifically, the molecular cargo inside astrocyte-derived EVs was evaluated to determine wither the glial-derived EV regulated survival and development of electrophysiological function in cultured human cortical neurons. EVs such as exosomes are generated as intraluminal vesicles through inward budding of the multivesicular body; a subtype of late stage endosomes.⁷⁻¹⁴ Endogenously programmed intracellular trafficking of multivesicular bodies can either fuse them with lysosomes for degradation, or with the plasma membrane to facilitate the release of their contents into the extracellular space in the form of EVs.¹⁵ In addition to the cellular ‘waste disposal’ role of EV, they are now regarded as major carriers of soluble factors that mediate paracrine communication between various types of cells within the CNS.¹⁶⁻¹8 EVs encapsulate transferrable mRNA, miRNA, lncRNA and numerous proteins and lipids in order to modulate recipient cell behavior, including protein translation, post-transcriptional gene regulation, and direct expression of transferred mRNAs.^{15,19}

The most common proteins encapsulated in EVs are related to vesicular transport and fusion (Rab GTPasas, SNAREs, annexins, and flotillin), different integrins and tetraspanins (CD63, CD9, CD81, and CD82), heat shock proteins (Hsc/Hsp 70 and 90), and proteins implicated in the biogenesis of multivesicular bodies.⁸ The role of astrocyte-derived EVs in different neuronal subtypes has been studied in several in vitro and murine model studies. These studies used neuroblastoma cell lines or murine derived neurons to investigate which specific cargo proteins or RNAs were present and their impact on cellular integrity, survival, functional maturity, and pathogenic development^{10, 20-22} Although the previous studies have significantly expanded the understanding of the complex role of glial-derived EVs on neurons, the murine-derived glial cells and neuroblastoma cell lines used may not adequately reflect glia and neuron interactions in human neural tissue and therefore present significant limitations when seeking to recapitulate the complex physiological environment of the human CNS. Moreover, previous studies that focused on glial-derived EVs have not shown their functional effect on altering neuronal electrophysiology, which is one of the most important criteria for determining major function of neurons.

In this study, human induced pluripotent stem cell (hiPSC)-derived cortical neurons and human astrocyte-derived EVs were used to investigate the effect of astrocyte-derived EVs on physiological behavior in human neurons, with a particular focus on their positive regulation of neuronal electrophysiology. The vesicular cargos encapsulated from the astrocytic cytoplasm enhanced the recipient neuron's survival and further support transmembrane ion channel protein activity to promote electrochemical maturation of neurons in culture. The data presented here demonstrate that astrocyte-derived EVs enhance survival of cortical neurons during differentiation, as evidenced by significantly lower expression of senescence and apoptosis markers at the early stages of culture. More importantly, optimal concentration of EV treatment on neurons dramatically enhanced their electrophysiological function in culture, indicating that the vesicular molecules delivered to neurons might have a critical effect on supporting the development of correct ion channel expression patterns.

The data herein contributes to improving the understanding of paracrine interactions between astrocytes and cortical neurons in the human brain. These data are important for future efforts to model CNS disease using human iPSC-derived cortical neurons, and will help facilitate study of the various pathogenic roles of glial cells in the human nervous system.

In certain preferred aspects, the disclosed systems can provide advances in understanding the physiological role of astrocyte-derived EVs on modulating the functional properties of human neurons. More specifically, among others, the following:
Human astrocyte-derived EVs are actively endocytosed by hiPSC-derived cortical neurons and endow anti-apoptotic effects to early stage, immature neurons that were forced to go through stressful differentiation process.
Optimal concentrations for EV treatments to cultured cortical neurons are defined in terms of their capacity to prolong cell viability and prevent early stage cell senescence in culture.
Cortical neurons that have taken up astrocyte-derived EVs exhibit significantly enhanced single cell electrophysiology, including resting membrane potential, action potential duration, speed of repolarization, and a remarkable increase in the percentage of cells capable of firing repetitive action potential trains in response to injected depolarizing current.

It is becoming increasingly important to reduce the significant gap that currently exists between neurophysiology observed in culture and that measured in the actual nervous system. This is especially true for enabling reliable modeling of human neuropathies that usually aren't neuron autonomous.

To apply nerve tissue modeling for efficient drug development, it can be important to have an understanding of how neurons survive and function with the aid of supporting cells. In that sense, recent advances have been made that improve our understanding of the crosstalk between neurons and glia from developmental to neurodegenerative states. Within this field, the work herein represents an important advance, especially in terms of understanding the comprehensive role of astroglia-derived EVs on human iPSC-derived neuron viability and electrophysiological function.

The results provide new insight into functional aspects of EVs as means of intercellular communication in the human nervous system, and drive important projects focused on the potential for EV-derived therapies to combat the onset and progression of neurodegenerative disease.

### Extracellular Vesicles and Adeno-Associated Virus (AAV)

Standard AAV vectors are generated with a producer cell line like the widely used 293T. During vector production, assembled AAV vector particles accumulate inside the producer cells and purification protocols often describe a process that begins with cell lysis to release AAV particles with subsequent purification steps.

AAV particles are often found with cellular exosomes, small membrane vesicles or microvesicles (approx. 40-150 nm diameter) that are secreted from the cellular membrane into the culture medium. AAV particles are either associated with the outside surface of the exosome or are carried inside the vesicle, which may also contain other cellular molecules such as proteins or nucleic acids. This conjunction of vectors and exosome was given the name vexosomes (vector-exosomes), exosome AAVs, or exo-AAVs. These particles have advantages over the conventionally purified AAV vectors, for example a higher transduction efficiency (the same number of AAV particles is able to infect more cells). Moreover, exo-AAVs were more resistant to neutralizing anti-AAV antibodies. This is particularly useful for therapeutic applications in vivo where endogenous anti-AAV antibodies often neutralize the therapeutic efficacy of AAV vectors.

miRNAs and Extracellular Vesicles (Exosomes)

Research shows that microRNAs (miRNAs) travel around the body contained within exosomes. MicroRNAs (miRNAs) are small non-coding RNAs that regulate gene expression. They exert their effects on the cells they are synthesized in, and are also released into the extracellular space and transported in body fluids such as blood and urine.

Recent research has uncovered evidence that miRNAs are transported in body fluids within exosomes; small cell-derived vesicles that function in intercellular communication processes. Once released into the extracellular fluid, exosomes fuse with other cells and transfer their cargo to the acceptor cell. Exosomal miRNAs may have important functions in cell-cell communication and have potential as biomarkers to detect and monitor disease. We review recent progress in this exciting research field.

miRNAs are Actively Sorted into Vesicles

Exosomal miRNA signatures also alter under specific conditions and diseases. For example, exosomal let-7f, miR-20b and miR-30e-3p levels were altered in the plasma of patients with small-cell lung cancer. Similarly, miR-21 and miR-141 have been found to be altered in the presence of benign tumors and ovarian cancer.

Exosomal miRNAs Control Gene Expression in Target Cells

Exosomes play an important role in information exchange between cells. Once released, they travel throughout the body before releasing their contents into a recipient cell. As miRNAs regulate gene expression, this raises the possibility that miRNAs can control gene expression in target cells.

A striking example of the control that exosomal miRNAs can have on a recipient cell gene expression has recently been uncovered in breast cancer exosomes. It was shown that cancer exosomes are able to rapidly silence certain mRNAs in recipient cells, see, e.g., Dilsiz N. Role of exosomes and exosomal microRNAs in cancer. Future Sci OA. 2020 Feb. 26; 6(4):FSO465, incorporated herein by reference in its entirety.

### Methods of Treatment

Provided herein are methods of preventing or treating a neuropathy in a patient. For example, the neuropathy is a traumatic peripheral nerve injury, chemotherapy induced peripheral neuropathy, traumatic brain injury, stroke, Charcot-Marie-Tooth disease (CMT), Amyotrophic Lateral Sclerosis (ALS), Parkinson's disease, Alzheimer's disease, frontotemporal dementia, Huntington's disease, Multiple Sclerosis, Congenital Myasthenia, Apraxia, or Hypertonia, myasthenia gravis, or spinal muscular atrophy.

The method comprises administering to the patient an effective amount of a pharmaceutical composition comprising the glial-derived extracellular vesicle (for example, the vesicle includes miRNA-21, miRNA-132, miRNA-9, miRNA-27a, miRNA-221, miRNA-200a-3p, miRNA-361, miRNA-274, miR-873a-5p, AAV1, AAV2, AAV4, AAV5, AAV6, AAV7, AAV8, AAV9 AAVDJ8, AAVrh10, glial derived neurotrophic factor, brain derived neurotrophic factor, Ciliary neurotrophic factor, GFAP). Additional exemplary cargos may be found at Goetzl E J, Mustapic M, Kapogiannis D, et al. Cargo proteins of plasma astrocyte-derived exosomes in Alzheimer's disease. FASEB J. 2016; 30(11):3853-3859, incorporated herein by reference in its entirety.

In embodiments, the patient is administered the composition intravenously. In embodiments, the glial-derived extracellular vesicle may be administered by intravenous, ultrasonic, or subcutaneous administration. Alternatively, the administration may be intraperitoneal, intramuscular, intra-articular, intraarterial, intrasynovial, intrasternal, intrathecal, intralesional and intracranial injection or infusion techniques. In embodiments, viral vectors described herein may be administered by intravenous administration, intramuscular administration, subcutaneous administration, or intrathecal administration.

The composition may be administered in a range from about 1×10⁹ to about 2×10⁹ genomic copies/mouse. Alternatively, the composition may be administered in an amount equivalent to a protein standard. The conversion of animal doses to human equivalent doses based on body surface area in shown in the table below (based on FDA Guidance, “Guidance for Industry Estimating the Maximum Safe Starting Dose in Initial Clinical Trials for Therapeutics in Adult Healthy Volunteers,” U.S. Department of Health and Human Services Food and Drug Administration Center for Drug Evaluation and Research (CDER) July 2005 Pharmacology and Toxicology, incorporated herein by reference in its entirety).

TABLE 1
The conversion of animal doses to human equivalent doses
Table 1: Conversion of Animal Doses to Human
Equivalent Doses Based on Body Surface Area

| | To Convert | To Convert Animal Dose in mg/kg | |
|---|---|---|---|
| | Animal Dose in | to HED^{a} in mg/kg, Either: | |
| | mg/kg to Dose in | Divide | Multiply |
| | mg/m², Multiply | Animal | Animal |
| Species | by kₘ | Dose By | Dose By |
| Human | 37 | — | — |
| Child (20 kg)^{b} | 25 | — | — |
| Mouse | 3 | 12.3 | 0.08 |
| Hamster | 5 | 7.4 | 0.13 |
| Rat | 6 | 6.2 | 0.16 |
| Ferret | 7 | 5.3 | 0.19 |
| Guinea pig | 8 | 4.6 | 0.22 |
| Rabbit | 12 | 3.1 | 0.32 |
| Dog | 20 | 1.8 | 0.54 |
| Primates: | | | |
| Monkeys^{c} | 12 | 3.1 | 0.32 |
| Marmoset | 6 | 6.2 | 0.16 |
| Squirrel monkey | 7 | 5.3 | 0.19 |
| Baboon | 20 | 1.8 | 0.54 |
| Micro-pig | 27 | 1.4 | 0.73 |
| Mini-pig | 35 | 1.1 | 0.95 |

In accordance with the methods described herein, a “subject in need of” is a subject having a neuropathy (neuropathic disease or disorder), or a subject having an increased risk of developing a neuropathy, relative to the population at large.

The effective amount for prevention or treatment of a neuropathy is from about 0.01 ng to about 10,000 nM of the composition. The composition comprises a concentration containing about, at least about, or at most about 0.01, 1.0, 10.0, 100, 200, 300, 400, 500, 600, 700, 800, 900, 1000, 2000, 3000, 4000, 5000, 6000, 7000, 8000, 9000, or 10000 nM, of glial-derived extracellular vesicles, or any range derivable therein.

The above numerical values may also be the dosage that is administered to the patient based on the patient's weight, expressed as ng/kg, mg/kg, or g/kg, and any range derivable from those values. The composition may have a concentration of exosomes that are 0.01, 1.0, 10.0, 100, 200, 300, 400, 500, 600, 700, 800, 900, or 1000 ng/ml, or any range derivable therein. The effective amount is from about 0.01 ng/mL to about 10,000 ng/mL of the composition.

In embodiments, the composition (including the glial-derived extracellular vesicle) may be administered to (or taken by) the patient 1, 2, 3, 4, 5, 6, 7, 8, 9, 10, 11, 12, 13, 14, 15, 16, 17, 18, 19, 20 or more times, or any range derivable therein, and they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months, or any range derivable therein.

In embodiments, the composition may be administered once daily, twice daily, three times daily, four times daily, five times daily, or six times daily (or any range derivable therein) and/or as needed to the patient.

In embodiments, the composition may be administered every 2, 4, 6, 8, 12 or 24 hours (or any range derivable therein) to or by the patient. In some embodiments, the patient is administered the composition for a certain period of time or with a certain number of doses.

In embodiments, the composition (including the glial-derived extracellular vesicle) is administered in an amount of 0.001 to 1000 mg/day. In embodiments, the composition is administered in a range from about 0.001 mg/kg to about 1000 mg/kg, about 0.01 mg/kg to about 100 mg/kg, about 10 mg/kg to about 250 mg/kg, about 0.1 mg/kg to about 15 mg/kg; or any range in which the low end of the range is any amount between 0.001 mg/kg and 900 mg/kg and the upper end of the range is any amount between 0.1 mg/kg and 1000 mg/kg (e.g., 0.005 mg/kg and 200 mg/kg, 0.5 mg/kg and 20 mg/kg). Effective doses will also vary, as recognized by those skilled in the art, depending on the diseases treated, route of administration, excipient usage, and the possibility of co-usage with other therapeutic treatments such as use of other agents.

In embodiments, methods comprising combination therapy are provided. As used herein, “combination therapy” or “co-therapy” includes the administration of a therapeutically effective amount of a pharmaceutical composition comprising the glial-derived extracellular vesicle, with at least one additional active agent, as part of a specific treatment regimen intended to provide a beneficial effect from the co-action of the active agents in the regimen, e.g. an additional neuropathic treatment. Exemplary additional therapies may include small molecules, gene therapy (e.g., gene therapy for ALS includes expression native TDP-43, FUS, C9ORF72) or cell therapy. In other examples, gene therapy for CMT (Charcot-Marie Tooth Disease Type 1 and/or Type 2) may be used in combination with the claimed invention, for example, Engensis (VM202) which is a plasmid DNA designed to simultaneously express two isoforms of hepatocyte growth factor (HGF), HGF 728 and HGF 723.

Table 2 below outlines FDA approved drugs for neuropathies that can be used as combination therapies (Vinik A, et al. Diabetic Neuropathies. [Updated 2018 Feb. 5]. MDText.com, Inc.; 2000-. Table 7, Drugs Approved by the FDA for Treatment of Neuropathic Pain Syndromes:

| | | Beginning | | Maximum | Duration of |
|---|---|---|---|---|---|
| Medication | Indication | Dosages | Titration | Dosage | Adequate Trial |
| Gabapentin | Postherpetic | 100-300 mg | Increase by | 3600 mg/d | 3-8 wk for |
| | neuralgia | every night or | 100-300 mg | (1200 mg 3×/d); | titration plus 1-2 |
| | | 100-300 mg | 3×/d every 1-7 d | reduce if | wk at maximum |
| | | 3×/d | as tolerated | low creatinine | tolerated |
| | | | | clearance | dosage |
| Pregabalin | DPN | 50 mg three | Increase up to | 600 mg a day | Start with 50 mg |
| | | times a day | 100 mg three | | TID and |
| | | | times a day | | increase upto |
| | | | | | 100 mg TID over |
| | | | | | 1 week |
| Lamotrigine | Postherpetic | 200-400 mg | Start with 25 to | 500 mg a day | 3 to 5 wk for |
| | neuralgia | every night. | 50 mg every | | titration ad 1-2 |
| | | | other day and | | wk at maximum |
| | | | increase by 25 | | tolerated |
| | | | mg every week. | | dosage. |
| Carbamazepine** | Trigeminal | 200 mg/d (100 | Add up to 200 | 1200 mg/d | |
| | neuralgia | mg bid) | mg/d in | | |
| | | | increments of | | |
| | | | 100 mg every | | |
| | | | 12 h | | |
| Duloxetine | DPN | 30 mg | 30 mg weekly | 120 mg | 2 wk |
| 5% lidocaine | Postherpetic | Maximum of 3 | None needed | Maximum of 3 | 2 wk |
| patch | neuralgia | patches daily for | | patches daily for | |
| | | a maximum of | | a maximum of | |
| | | 12 hr | | 12 hr | |
| Opioid | Moderate to | 5-15 mg every 4 | After 1-2 wk, | No maximum | 4-6 wk |
| analgesics* | severe pain | hr as needed | convert total | with careful | |
| | | | daily dosage to | titration; | |
| | | | long-acting | consider | |
| | | | medication as | evaluation by | |
| | | | needed | pain specialist | |
| | | | | at dosages | |
| | | | | exceeding 120- | |
| | | | | 180 mg/d | |
| Tramadol | Moderate to | 50 mg | Increased by | 400 mg/d (100 | 4 wk |
| hydrochloride | moderately | 1 or 2×/d | 50-100 mg/d in | mg 4×/d); in | |
| | severe pain | | divided doses | patients older | |
| | | | every 3-7 d as | than 75 yr, 300 | |
| | | | tolerated | mg/d in divided | |
| | | | | doses | |
| Tricyclic | Chronic pain | 10-25 mg every | Increase by 10- | 75-150 mg/d; if | 6-8 wk with at |
| antidepressants | | night | 25 mg/d every | blood level of | least 1-2 wk at |
| (eg, nortriptyline | | | 3-7 d as | active drug and | maximum |
| hydrochloride or | | | tolerated | its metabolite | tolerated |
| desipramine | | | | is <100 ng/mL, | dosage |
| hydrochloride) | | | | continue | |
| | | | | titration with | |
| | | | | caution | |
| Duloxetine | Diabetic | 30 mg bid | Increase by 60 | 120 mg/d | 4 wk |
| Serotonin/norepinephrine | neuropathic | | to 60 bid. No | | |
| Reuptake | pain | | further titration | | |
| inhibitor | | | | | |
| Fluoxetine | Diabetic | 30 mg bid | Increase by 60 | 120 mg/d | 4 wk |
| Serotonin/norepinephrine | neuropathic | | to 60 bid. No | | |
| Reuptake | pain | | further titration | | |
| inhibitor | | | | | |
| Tapentadol ER | Diabetic | 50 mg bid | Increase by 50 | 500 mg/d | |
| | neuropathic | | mg/bid every 3 | | |
| | pain | | days as | | |
| | | | tolerated | | |

### Therapy for Charcot-Marie Tooth Disease (CMT)

In other examples, the combination therapy for CMT may include administration of a therapeutically effective amount of a pharmaceutical composition comprising the glial-derived extracellular vesicle, with at least one additional treatment regimen intended to provide a beneficial effect from the co-action of the active agents in the regimen. The additional treatment may include physical and occupational therapies, braces and other orthopedic devices, and orthopedic surgery can help people cope with the disabling symptoms of the disease. In addition, pain-relief drugs can be prescribed for individuals who have severe nerve pain. Other cell-based therapies may include Mesenchymal stem cell (MSC) or hematopoietic stem cell transplants. Additional exemplary therapies are described in Morena J, Gupta A, Hoyle J C. Charcot-Marie-Tooth: From Molecules to Therapy. Int J Mol Sci. 2019; 20(14):3419. Published 2019 Jul. 12, incorporated herein by reference in its entirety.

### Gene Therapy for Targeted Delivery

CRISPR-based gene editing strategies targeting genetic neuropathies (such as ALS and Charcot Marie Tooth disease) are currently in development as a means to correct inheritable genetic mutations that cause these severe neurodegenerative conditions. For example, trials are currently ongoing for the use of gene therapy to restore SMN2 expression in patients with spinal muscular atrophy, see, for example Clinical Trial No: NCT03837184. Similarly, a number of gene correction strategies have shown promise in academic settings as a means to correct genetic mutations in ALS and CMT patients. See, e.g., Mol Ther Nucleic Acids. 2019 Jun. 7; 16:26-37. Epub 2019 Feb. 11 and Brain. 2019 May 1; 142(5):1227-1241, each of which is incorporated herein by reference in its entirety.

Combination therapies involving such therapeutic strategies with EV mediated treatments could involve simultaneous or sequential treatment of patients with a gene therapy vector and EVs containing additional trophic support factors to further enhance recovery. Alternatively, EVs could be used as a means to facilitate targeted delivery of the CRISPR-based or alternative gene editing machinery to neurons. Use of cell type-specific targeting moieties on the EV surface could be used to ensure delivery of the gene editing machinery only to the cell type of interest, thereby limiting the risk of off-target editing and/or unpredictable results in non-target tissues.

For example, the glial-derived exosome (or glial-derived extracellular vesicle) may be used to deliver the gene editing tools to the target cell of interest, e.g., to restore, or decrease expression of a specific protein of interest.

For example, the gene editing tools may include a gene editing protein (zinc finger nuclease (ZFN), a transcription activator-like effector nuclease (TALEN), a MegaTAL, a Cas protein, a Cre recombinase, a Hin recombinase, or a Flp recombinase), an RNA molecule and/or a ribonucleoprotein (RNP). For example, the RNP may include a Cas protein and a gRNA or a crRNA and a tracrRNA.

In some embodiments, the Cas protein is a Cas9 protein or a mutant thereof. Exemplary Cas proteins (including Cas9 and non-limiting examples of Cas9 mutants) are described below.

In other examples, the gene editing composition induces single-strand or doublestrand breaks in DNA within the cells. In some embodiments the gene editing composition further comprises a repair template polynucleotide.

Non-limiting descriptions relating to gene editing (including repair templates) using the CRISPR-Cas system are discussed in Ran et al. (2013) Nat Protoc. 2013 November; 8(11): 2281-2308, the entire content of which is incorporated herein by reference. Embodiments involving repair templates are not limited to those comprising the CRISPR-Cas system.

Non-limiting examples of Cas proteins include Cas1, Cas1B, Cas2, Cas3, Cas4, Cas5, Casio, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2 and in the NCBI database as under accession number Q99ZW2.1. UniProt database accession numbers A0A0G4DEU5 and CDJ55032 provide another example of a Cas9 protein amino acid sequence. Another non-limiting example is a Streptococcus thermophilus Cas9 protein, the amino acid sequence of which may be found in the UniProt database under accession number Q03116.1. In some embodiments, the unmodified CRISPR enzyme has DNA cleavage activity, such as Cas9. In certain embodiments the CRISPR enzyme is Cas9, and may be Cas9 from S pyogenes or S. pneumoniae. In various embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence. For example, an aspartate-to-alanine substitution in the RuvC I catalytic domain of Cas9 from S. pyogenes converts Cas9 from a nuclease that cleaves both strands to a nickase (cleaves a single strand). Other examples of mutations that render Cas9 a nickase include, without limitation, II840A, N854A, and N863A. In aspects of the invention, nickases may be used for genome editing via homologous recombination.

Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR enzyme corresponding to the most frequently used codon for a particular amino acid.

In other examples, EVs could be used as a means to facilitate targeted delivery of the CRISPR-based or alternative gene editing machinery to neurons for CMT. CMT is a heterogeneous disease and mutations associated with it can occur in several different genes. Depending on the gene affected, CMT is divided into several types and subtypes. The most common cause of CMT is an duplication of a PMP22 gene. Other mutations affect the MFN2 gene on, which encodes a mitochondrial protein preventing fusion or aggregation of itself. This causes mitochondria cannot move along the axon toward the synapse. Exemplary CMT mutations that could be addressed using gene editing tools described herein (using glial derived extracellular vesicles or glial-derived exosomes) include CMT type 1 mutation—CMT1A (PMP22 mutation), CMT1B (MPZ mutation), CMT1C (LITAF mutation), CMT1D (EGR2 mutation), CMT1E (PMP22 mutation), or CMT type 2 mutation—CMT2A (MFN2 mutation), CMT2B (RAB7A mutation), CMT2C (TRPV4 mutation), CMT2D (GARS mutation), CMT2E (NEFL mutation), CMT2F (HSPB1 mutation), CMT2G (12q12-q13 mutation), CMT2H (GDAP1 mutation), CMT2L (HSPB8 mutation), CMT2N (AARS mutation), CMT2M (DMN mutation).

### Functional Maturation

As shown herein, the technology is derived from the unique combination and treatment conditions of glial cell-derived EVs for neuronal culture viability and functionality. In other examples, this technology is relevant to the induced pluripotent stem cells, cellular reprogramming, and iPSC markets for cardiomyocytes, hepatocytes, neurons, and other cell types.

With respect to therapeutic agents, the beneficial effect of the combination includes, but is not limited to, pharmacokinetic or pharmacodynamic co-action resulting from the combination of therapeutically active compounds. With respect to non-therapeutic agents, the beneficial effect of the combination may relate to the mitigation of toxicity, side effect, or adverse event associated with a therapeutically active agent in the combination.

Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

### Kits

In aspects, a kit including the glial-derived extracellular vesicle and reagents is provided.

In embodiments, the glial-derived extracellular vesicle in the kit is suitable for delivery (e.g., local injection) to a subject.

The present invention also provides packaging and kits comprising pharmaceutical compositions for use in the methods of the present invention. The kit can comprise one or more containers selected from the group consisting of a bottle, a vial, an ampoule, a blister pack, and a syringe.

The kit can further include one or more of instructions for use in treating and/or preventing a disease, condition or disorder of the present invention (e.g., a neuropathic disease or disorder), one or more syringes, one or more applicators, or a sterile solution suitable for reconstituting a pharmaceutical composition of the present invention.

### General Definitions

The following definitions are included for the purpose of understanding the present subject matter and for constructing the appended patent claims. The abbreviations used herein have their conventional meanings within the chemical and biological arts.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

The term “disease” refers to any deviation from the normal health of a mammal and includes a state when disease symptoms are present, as well as conditions in which a deviation (e.g., a neuropathy, specifically a PNS neuropathy) has occurred, but symptoms are not yet manifested.

The terms “neuroinjury” and “injury” are often used interchangeably herein. “neurotrauma” is one embodiment of a “neuroinjury” and may generally be considered a synonym. A “neuroinjury” is an injury that causes some destruction or death of neurological tissue. A neuroinjury generally has as sequelae some loss, e.g., a diminution of mental, sensory or muscle function. Thus the compositions described herein may be used to treat or prevent a neuroinjury. For example, traumatic injury can cause severe stress and an apoptotic response in neurons. The data herein indicated that EVs inhibit apoptosis and thus may be beneficial in treating physical trauma to the CNS.

“Patient” or “subject in need thereof” refers to a living member of the animal kingdom suffering from or who may suffer from the indicated disorder. In embodiments, the subject is a member of a species comprising individuals who may naturally suffer from the disease. In embodiments, the subject is a mammal. Non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. In embodiments, the subject is a human.

The terms “subject,” “patient,” “individual,” etc. are not intended to be limiting and can be generally interchanged. That is, an individual described as a “patient” does not necessarily have a given disease, but may be merely seeking medical advice.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The term “exogenous” refers to a protein with which the cell or exosome is not normally associated or expresses in its native or wild type state.

As used herein, fusion proteins, also known as chimeric proteins, are proteins created through the joining of two or more genes which originally coded for separate proteins. Translation of this fusion gene results in a single polypeptide with function properties derived from each of the original proteins. Recombinant fusion proteins can be created artificially by recombinant DNA technology for use in biological research or therapeutics. Chimeric mutant proteins occur naturally when a large-scale mutation, typically a chromosomal translocation, creates a novel coding sequence containing parts of the coding sequences from two different genes. The functionality of fusion proteins is made possible by the fact that many protein functional domains are modular. In other words, the linear portion of a polypeptide which corresponds to a given domain, such as a tyrosine kinase domain, may be removed from the rest of the protein without destroying its intrinsic enzymatic capability.

A recombinant fusion protein is a protein created through genetic engineering of a fusion gene. This typically involves removing the stop codon from a cDNA sequence coding for the first protein, then appending the cDNA sequence of the second protein in frame through ligation or overlap extension PCR. That DNA sequence will then be expressed by a cell as a single protein. The protein can be engineered to include the full sequence of both original proteins, or only a portion of either.

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

As used in the description herein and throughout the claims that follow, the meaning of “a,” “an,” and “the” includes plural reference unless the context clearly dictates otherwise.

As used herein, “treating” or “treatment” of a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean inhibiting the progression of the condition, disorder or disease, slowing the progression of the condition, disorder or disease temporarily, although in some instances, it involves halting the progression of the condition, disorder or disease permanently.

As used herein, the terms “treat” and “prevent” are not intended to be absolute terms. In various embodiments, treatment can refer to a 10%, 20%, 30%, 40%, 50%, 60%, 70%, 80%, 90%, or 100% reduction in the severity of an established disease, condition, or symptom of the disease or condition. In embodiments, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. In embodiments, references to decreasing, reducing, or inhibiting include a change of 10%, 20%, 30%, 40%, 50%, 60%, 70%, 80%, 90% or greater as compared to a control level and such terms can include but do not necessarily include complete elimination. In embodiments, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

The terms “effective amount,” “effective dose,” etc. refer to the amount of an agent that is sufficient to achieve a desired effect, as described herein. In embodiments, the term “effective” when referring to an amount of cells or a therapeutic compound may refer to a quantity of the cells or the compound that is sufficient to yield an improvement or a desired therapeutic response without undue adverse side effects (such as toxicity, irritation, or allergic response) commensurate with a reasonable benefit/risk ratio when used in the manner of this disclosure. In embodiments, the term “effective” when referring to the generation of a desired cell population may refer to an amount of one or more compounds that is sufficient to result in or promote the production of members of the desired cell population, especially compared to culture conditions that lack the one or more compounds.

As used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (RNA or DNA) is free of the genes or sequences that flank it in its naturally-occurring state. Purified also defines a degree of sterility that is safe for administration to a human subject, e.g., lacking infectious or toxic agents.

In certain examples, the EV collection method used is based on centrifugal force to remove other particles (cell debris, apoptotic bodies, and the like), and is at least 80%, 85%, 90%, 95%, 96%, 97%, 98%, or 99% pure. The extracellular vesicles can also be substantially purified. The term “substantially purified” as used herein may refer to extracellular vesicles or exosomes that are substantially enriched in a sample. The sample can be substantially purified or enriched for the extracellular vesicles or exosomes of interest such that the sample is at least about 50%, 60%, 70%, 80%, 85%, 90%, 95%, 99% or greater of the desired extracellular vesicles or exosomes or less than about 40%, 30%, 20%, 15%, 14%, 13%, 12%, 11%, 10%, 9%, 8%, 7%, 6%, 5%, 4%, 3%, 2%, 1% or less of the undesirable or other extracellular vesicles or exosomes present. In an exemplary embodiment, a composition includes substantially purified extracellular vesicle. In other embodiments, the composition includes a substantially purified exosome. In other examples, the composition includes a substantially purified glial-derived extracellular vesicle and at least one substantially purified glial-derived exosome. Another embodiment is directed to a pharmaceutical composition for treating or preventing a neuropathy in a subject including a therapeutically effective amount of substantially purified glial-derived extracellular vesicle or a glial-derived exosome.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

A “control” sample or value refers to a sample that serves as a reference, usually a known reference, for comparison to a test sample. For example, a test sample can be taken from a test subject, e.g., a subject with a neuropathy, and compared to samples from known conditions, e.g., a subject (or subjects) that does not have a neuropathy (a negative or normal control), or a subject (or subjects) who does have a neuropathy (positive control). A control can also represent an average value gathered from a number of tests or results. One of skill in the art will recognize that controls can be designed for assessment of any number of parameters. One of skill in the art will understand which controls are valuable in a given situation and be able to analyze data based on comparisons to control values. Controls are also valuable for determining the significance of data. For example, if values for a given parameter are variable in controls, variation in test samples will not be considered as significant.

The term, “normal amount” with respect to a compound (e.g., a protein or mRNA) refers to a normal amount of the compound in an individual who does not have a neuropathy in a healthy or general population. The amount of a compound can be measured in a test sample and compared to the “normal control” level, utilizing techniques such as reference limits, discrimination limits, or risk defining thresholds to define cutoff points and abnormal values (e.g., for a neuropathy or a symptom thereof). The normal control level means the level of one or more compounds or combined compounds typically found in a subject known not suffering from a neuropathy Such normal control levels and cutoff points may vary based on whether a compounds is used alone or in a formula combining with other compounds into an index. Alternatively, the normal control level can be a database of compounds patterns from previously tested subjects who did not develop a neuropathy or a particular symptom thereof (e.g., in the event the a neuropathy develops or a subject already having a neuropathy is tested) over a clinically relevant time horizon.

The level that is determined may be the same as a control level or a cut off level or a threshold level, or may be increased or decreased relative to a control level or a cut off level or a threshold level. In some aspects, the control subject is a matched control of the same species, gender, ethnicity, age group, smoking status, body mass index (BMI), current therapeutic regimen status, medical history, or a combination thereof, but differs from the subject being diagnosed in that the control does not suffer from the disease (or a symptom thereof) in question or is not at risk for the disease.

Relative to a control level, the level that is determined may an increased level. As used herein, the term “increased” with respect to level (e.g., protein or mRNA level) refers to any % increase above a control level. In various embodiments, the increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, relative to a control level.

Relative to a control level, the level that is determined may a decreased level. As used herein, the term “decreased” with respect to level (e.g., protein or mRNA level) refers to any % decrease below a control level. In various embodiments, the decreased level may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, relative to a control level.

The terms “polypeptide,” “peptide” and “protein” are used interchangeably herein to refer to a polymer of amino acid residues, wherein the polymer may in embodiments be conjugated to a moiety that does not consist of amino acids. The terms also apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed or chemically synthesized as a single moiety.

“Polypeptide fragment” refers to a polypeptide that has an amino-terminal and/or carboxy-terminal deletion, in which the remaining amino acid sequence is usually identical to the corresponding positions in the naturally-occurring sequence. Fragments typically are at least 5, 6, 8 or 10 amino acids long, at least 14 amino acids long, at least 20 amino acids long, at least 50 amino acids long, or at least 70 amino acids long.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. In embodiments, the percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “identical” or percent “identity,” in the context of two or more nucleic acids or polypeptide sequences, refer to two or more sequences or subsequences that are the same or have a specified percentage of amino acid residues or nucleotides that are the same (e.g., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire polypeptide sequence or an individual domain thereof), when compared and aligned for maximum correspondence over a comparison window, or designated region as measured using a sequence comparison algorithm or by manual alignment and visual inspection. In embodiments, two sequences are 100% identical. In embodiments, two sequences are 100% identical over the entire length of one of the sequences (e.g., the shorter of the two sequences where the sequences have different lengths). In embodiments, identity may refer to the complement of a test sequence. In embodiments, the identity exists over a region that is at least about 10 to about 100, about 20 to about 75, about 30 to about 50 amino acids or nucleotides in length. In embodiments, the identity exists over a region that is at least about 50 amino acids or nucleotides in length, or more preferably over a region that is 100 to 500, 100 to 200, 150 to 200, 175 to 200, 175 to 225, 175 to 250, 200 to 225, 200 to 250 or more amino acids or nucleotides in length.

For sequence comparison, typically one sequence acts as a reference sequence, to which test sequences are compared. In embodiments, when using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window” refers to a segment of any one of the number of contiguous positions (e.g., least about 10 to about 100, about 20 to about 75, about 30 to about 50, 100 to 500, 100 to 200, 150 to 200, 175 to 200, 175 to 225, 175 to 250, 200 to 225, 200 to 250) in which a sequence may be compared to a reference sequence of the same number of contiguous positions after the two sequences are optimally aligned. In embodiments, a comparison window is the entire length of one or both of two aligned sequences. In embodiments, two sequences being compared comprise different lengths, and the comparison window is the entire length of the longer or the shorter of the two sequences. In embodiments relating to two sequences of different lengths, the comparison window includes the entire length of the shorter of the two sequences. In embodiments relating to two sequences of different lengths, the comparison window includes the entire length of the longer of the two sequences.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Non-limiting examples of algorithms that are suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 may be used, with the parameters described herein, to determine percent sequence identity for nucleic acids and proteins. Software for performing BLAST analyses is publicly available through the National Center for Biotechnology Information (NCBI), as is known in the art. An exemplary BLAST algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. In embodiments, the NCBI BLASTN or BLASTP program is used to align sequences. In embodiments, the BLASTN or BLASTP program uses the defaults used by the NCBI. In embodiments, the BLASTN program (for nucleotide sequences) uses as defaults: a word size (W) of 28; an expectation threshold (E) of 10; max matches in a query range set to 0; match/mismatch scores of 1,−2; linear gap costs; the filter for low complexity regions used; and mask for lookup table only used. In embodiments, the BLASTP program (for amino acid sequences) uses as defaults: a word size (W) of 3; an expectation threshold (E) of 10; max matches in a query range set to 0; the BLOSUM62 matrix (see Henikoff & Henikoff, Proc. Natl. Acad. Sci. USA 89:10915 (1992)); gap costs of existence: 11 and extension: 1; and conditional compositional score matrix adjustment.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

“Nucleic acid” refers to nucleotides (e.g., deoxyribonucleotides, ribonucleotides, and 2′-modified nucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent, or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

“Operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences.

A “spacer” as used herein refers to a peptide that joins the proteins comprising a fusion protein. Generally a spacer has no specific biological activity other than to join the proteins or to preserve some minimum distance or other spatial relationship between them. However, the constituent amino acids of a spacer may be selected to influence some property of the molecule such as the folding, net charge, or hydrophobicity of the molecule.

The term “vector” or “construct” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element).

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides and/or ribonucleotides, and/or analogs, derivatives or modifications thereof. Different polynucleotides may have different three-dimensional structures, and may perform various functions, known or unknown. Non-limiting examples of polynucleotides include genomic DNA, a genome, mitochondrial DNA, a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

The term “amino acid residue,” as used herein, encompasses both naturally-occurring amino acids and non-naturally-occurring amino acids. Examples of non-naturally occurring amino acids include, but are not limited to, D-amino acids (i.e. an amino acid of an opposite chirality to the naturally-occurring form), N-α-methyl amino acids, C-α-methyl amino acids, β-methyl amino acids and D- or L-β-amino acids. Other non-naturally occurring amino acids include, for example, β-alanine (β-Ala), norleucine (Nle), norvaline (Nva), homoarginine (Har), 4-aminobutyric acid (γ-Abu), 2-aminoisobutyric acid (Aib), 6-aminohexanoic acid (ε-Ahx), ornithine (orn), sarcosine, α-amino isobutyric acid, 3-aminopropionic acid, 2,3-diaminopropionic acid (2,3-diaP), D- or L-phenylglycine, D-(trifluoromethyl)-phenylalanine, and D-p-fluorophenylalanine.

As used herein, “peptide bond” can be a naturally-occurring peptide bond or a non-naturally occurring (i.e. modified) peptide bond. Examples of suitable modified peptide bonds are well known in the art and include, but are not limited to, —CH₂NH—, —CH₂S—, —CH₂CH₂—, —CH═CH— (cis or trans), —COCH₂—, —CH(OH)CH₂—, —CH₂SO—, —CS—NH— and —NH—CO— (i.e. a reversed peptide bond) (see, for example, Spatola, Vega Data Vol. 1, Issue 3, (1983); Spatola, in Chemistry and Biochemistry of Amino Acids Peptides and Proteins, Weinstein, ed., Marcel Dekker, New York, p. 267 (1983); Morley, J. S., Trends Pharm. Sci. pp. 463-468 (1980); Hudson et al., Int. J. Pept. Prot. Res. 14:177-185 (1979); Spatola et al., Life Sci. 38:1243-1249 (1986); Hann, J. Chem. Soc. Perkin Trans. I 307-314 (1982); Almquist et al., J. Med. Chem. 23:1392-1398 (1980); Jennings-White et al., Tetrahedron Lett. 23:2533 (1982); Szelke et al., EP 45665 (1982); Holladay et al., Tetrahedron Lett. 24:4401-4404 (1983); and Hruby, Life Sci. 31:189-199 (1982)).

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides

### Pharmaceutical Compositions and Formulations

The present invention provides pharmaceutical compositions comprising an effective amount of a composition (e.g., a composition comprising the glial-derived extracellular vesicle) and at least one pharmaceutically acceptable excipient or carrier, wherein the effective amount is as described above in connection with the methods of the invention.

In one embodiment, the composition (e.g., a composition comprising the glial-derived extracellular vesicle) is further combined with at least one additional therapeutic agent in a single dosage form. In one embodiment, the at least one additional therapeutic agent comprises gabapentin, pregabalin, lamotrigine, carbamazepine, duloxetine, lidocaine, opioid analgesics, tramadol hydrochloride, tricyclic antidepressants (nortriptyline hydrochloride or desipramine hydrochloride), duloxetine (serotonin/norepinephrine reuptake inhibitor), fluoxetine, or tapentadol.

The term “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. Examples of pharmaceutically acceptable excipients include, without limitation, sterile liquids, water, buffered saline, ethanol, polyol (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, or suitable mixtures thereof.

A pharmaceutical composition can be provided in bulk or in dosage unit form. It is especially advantageous to formulate pharmaceutical compositions in dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved. A dosage unit form can be an ampoule, a vial, a suppository, a dragee, a tablet, a capsule, an IV bag, or a single pump on an aerosol inhaler.

In therapeutic applications, the dosages vary depending on the agent, the age, weight, and clinical condition of the recipient patient, and the experience and judgment of the clinician or practitioner administering the therapy, among other factors affecting the selected dosage. Generally, the dose should be a therapeutically effective amount. Dosages can be provided in mg/kg/day units of measurement (which dose may be adjusted for the patient's weight in kg, body surface area in m², and age in years). Exemplary doses and dosages regimens for the compositions in methods of treating muscle diseases or disorders are described herein.

The pharmaceutical compositions can take any suitable form (e.g, liquids, aerosols, solutions, inhalants, mists, sprays; or solids, powders, ointments, pastes, creams, lotions, gels, patches and the like) for administration by any desired route (e.g, pulmonary, inhalation, intranasal, oral, buccal, sublingual, parenteral, subcutaneous, intravenous, intramuscular, intraperitoneal, intrapleural, intrathecal, transdermal, transmucosal, rectal, and the like). For example, a pharmaceutical composition of the invention may be in the form of an aqueous solution or powder for aerosol administration by inhalation or insufflation (either through the mouth or the nose), in the form of a tablet or capsule for oral administration; in the form of a sterile aqueous solution or dispersion suitable for administration by either direct injection or by addition to sterile infusion fluids for intravenous infusion; or in the form of a lotion, cream, foam, patch, suspension, solution, or suppository for transdermal or transmucosal administration.

In embodiments, the pharmaceutical composition comprises an injectable form.

A pharmaceutical composition can be in the form of an orally acceptable dosage form including, but not limited to, capsules, tablets, buccal forms, troches, lozenges, and oral liquids in the form of emulsions, aqueous suspensions, dispersions or solutions. Capsules may contain mixtures of a compound of the present invention with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc.

A pharmaceutical composition can be in the form of a sterile aqueous solution or dispersion suitable for parenteral administration. The term parenteral as used herein includes subcutaneous, intracutaneous, intravenous, intramuscular, intra-articular, intraarterial, intrasynovial, intrasternal, intrathecal, intralesional and intracranial injection or infusion techniques.

A pharmaceutical composition can be in the form of a sterile aqueous solution or dispersion suitable for administration by either direct injection or by addition to sterile infusion fluids for intravenous infusion, and comprises a solvent or dispersion medium containing, water, ethanol, a polyol (e.g., glycerol, propylene glycol and liquid polyethylene glycol), suitable mixtures thereof, or one or more vegetable oils. Solutions or suspensions of the compound of the present invention as a free base or pharmacologically acceptable salt can be prepared in water suitably mixed with a surfactant. Examples of suitable surfactants are given below. Dispersions can also be prepared, for example, in glycerol, liquid polyethylene glycols and mixtures of the same in oils.

The pharmaceutical compositions for use in the methods of the present invention can further comprise one or more additives in addition to any carrier or diluent (such as lactose or mannitol) that is present in the formulation. The one or more additives can comprise or consist of one or more surfactants. Surfactants typically have one or more long aliphatic chains such as fatty acids which enables them to insert directly into the lipid structures of cells to enhance drug penetration and absorption. An empirical parameter commonly used to characterize the relative hydrophilicity and hydrophobicity of surfactants is the hydrophilic-lipophilic balance (“HLB” value). Surfactants with lower HLB values are more hydrophobic, and have greater solubility in oils, while surfactants with higher HLB values are more hydrophilic, and have greater solubility in aqueous solutions. Thus, hydrophilic surfactants are generally considered to be those compounds having an HLB value greater than about 10, and hydrophobic surfactants are generally those having an HLB value less than about 10. However, these HLB values are merely a guide since for many surfactants, the HLB values can differ by as much as about 8 HLB units, depending upon the empirical method chosen to determine the HLB value. All percentages and ratios used herein, unless otherwise indicated, are by weight.

### EXAMPLES

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1: Differentiation of Human Cortical Neurons from Neurogenin 2 Transgene Transduced hiPSCs

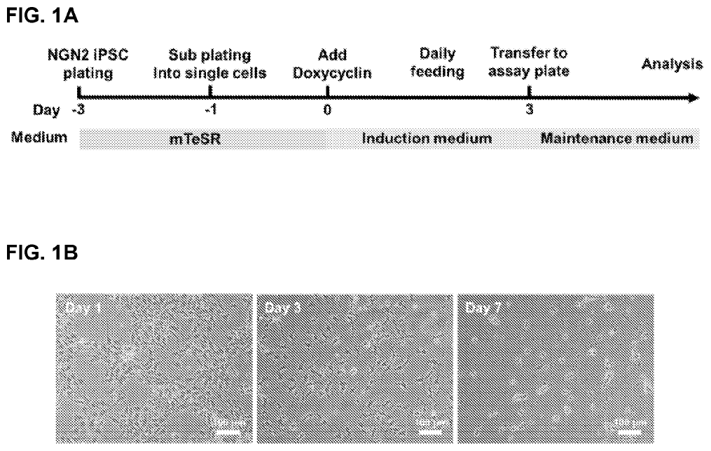

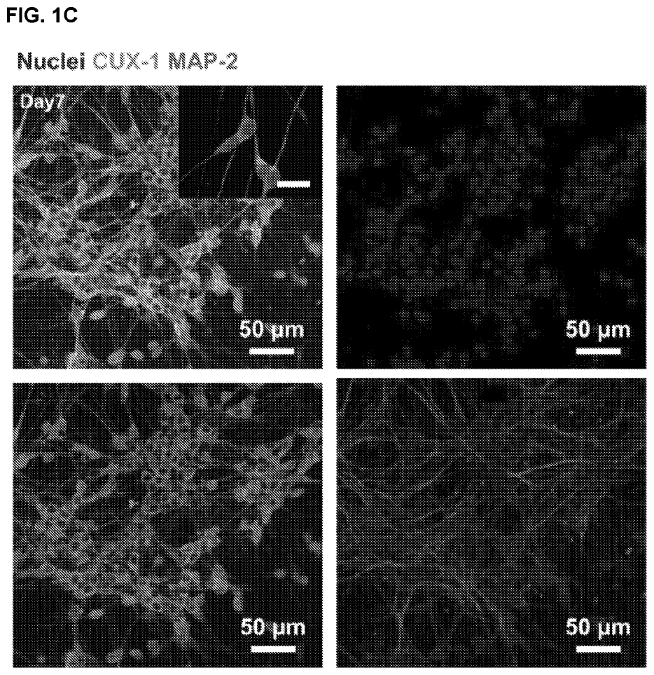

In vitro applications for human iPSC-derived neurons have been limited due to their heterogeneity and electrophysiological immaturity, coupled with high production costs and slow differentiation speeds. To address this issue, the effect of glial cell-derived EVs when applied to immature neurons rapidly differentiated from human iPSCs was investigated. Wang et. al. previously developed an iPSC line that harbored a doxycycline-inducible neurogenin 2 transgene; a transcription factor that drives rapid conversion of stem cells to cortical neurons. ^{23,24} In these cells, neuronal differentiation is induced by applying doxycycline for 24 hours after single cell dissociation of iPSCs and completed by exposing the early stage neurons to a specific induction medium for an additional 48 hours. This is then followed by a change to medium supplemented with BDNF, B27, laminin for maintenance after day 3 (FIG. 1A).

In the first 24 hour period following induction, the stem cells rapidly became polarized and began to exhibit growth cones indicative of initial neurite outgrowth. By day 3, neurite growth was more apparent and distinct axon outgrowth in each cell was observed. Cells at day 7 gained a typical neuronal morphology characterized by extremely long axons and dendrites approaching those of adjacent cells and small, well-defined soma (FIG. 1B). The differentiated neurons were transferred to laminin-coated assay plates at day 3 for analysis by immunocytochemistry or kept for further maturation. The overall differentiation period was dramatically shorter than that required in conventional human iPSC-derived neuron differentiation, which can take 4-6 weeks. ²⁵⁻²⁷ Immunocytochemistry data collected from these cells demonstrated a high degree of homogeneity in the cortical neurons generated from each experimental batch (n=5), indicating significant enhancements in reproducibility of human neuron differentiation. In particular, the uniform expression of CUX-1 observed in the majority of examined cells verified the successful generation of pyramidal cortical neurons specific to layers 2 and 3 of the cerebral cortex. The immunocytochemistry results highlight the reliability of the described method for producing differentiated neurons with which to study the effect of internalized EVs (FIG. 1C).

### Example 2: Human Astrocyte-Derived EV Collection and Characterization

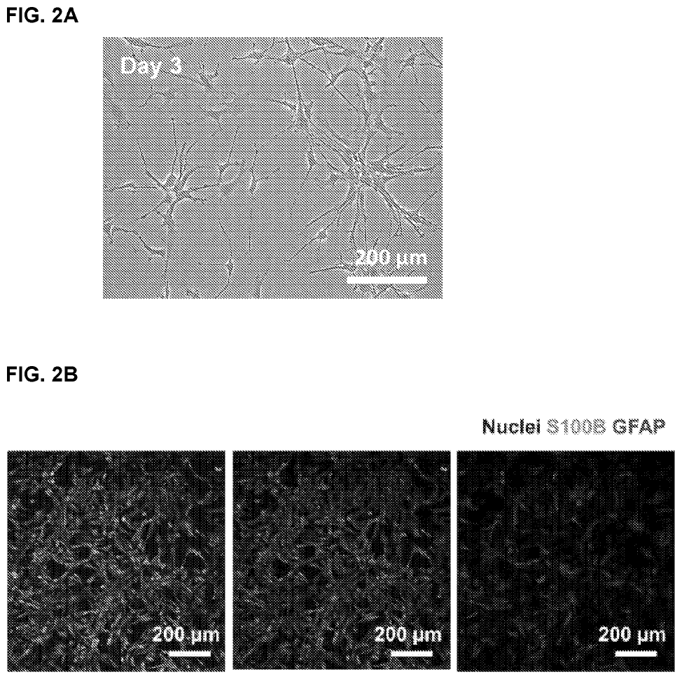

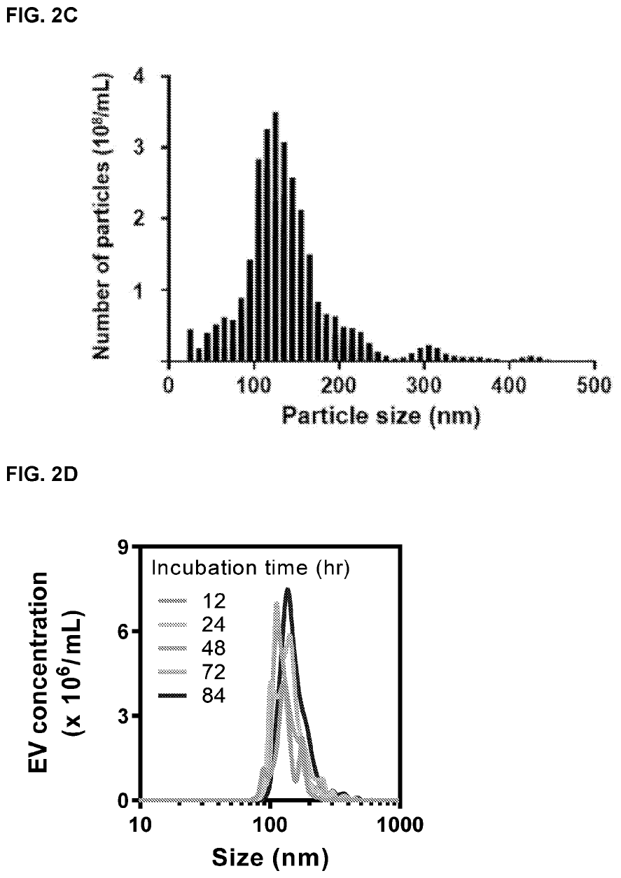

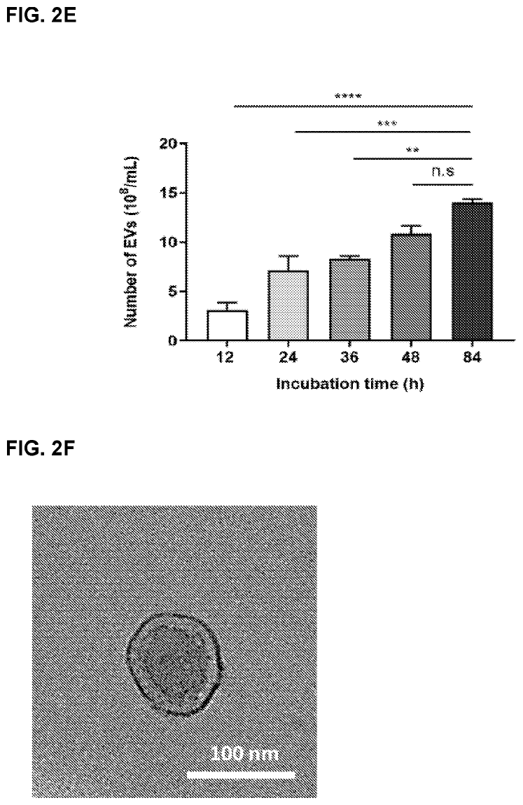

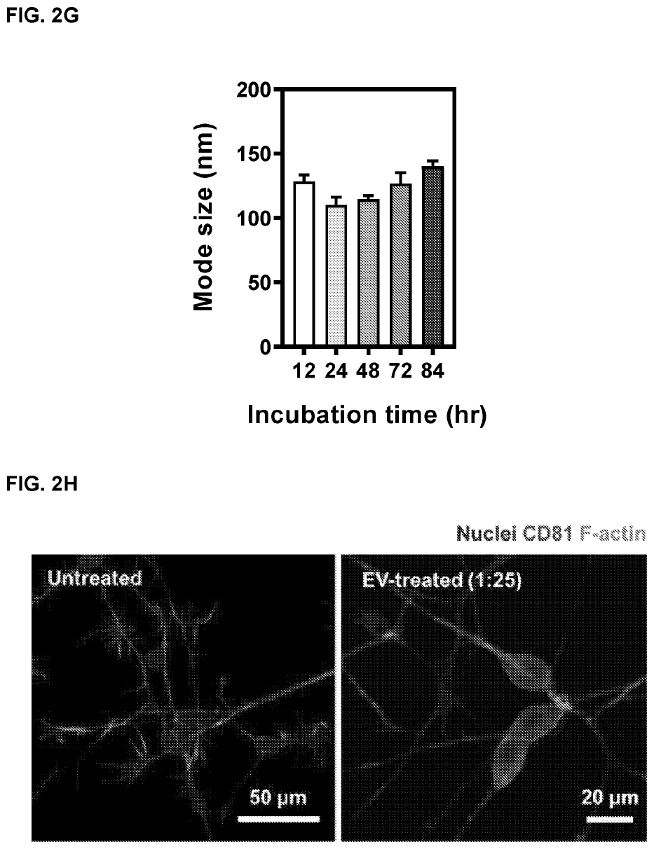

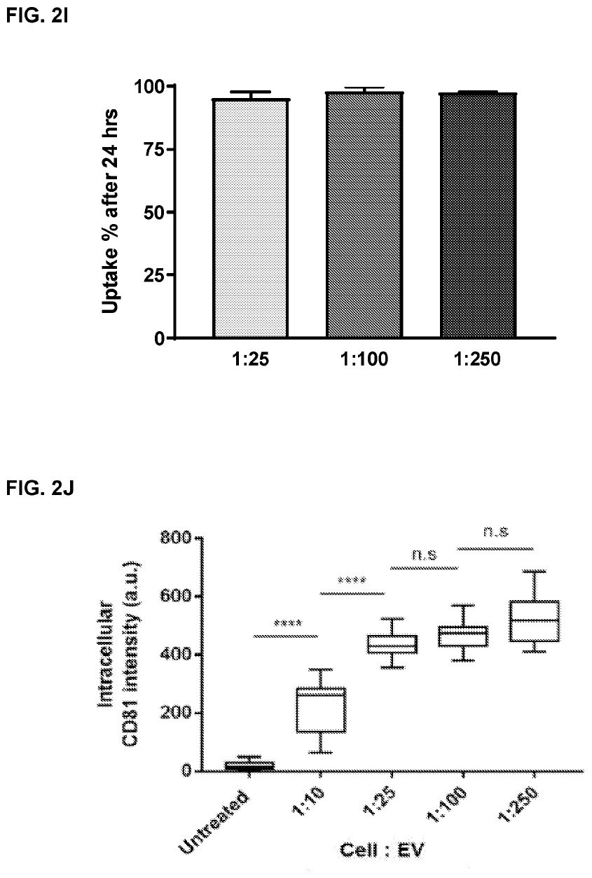

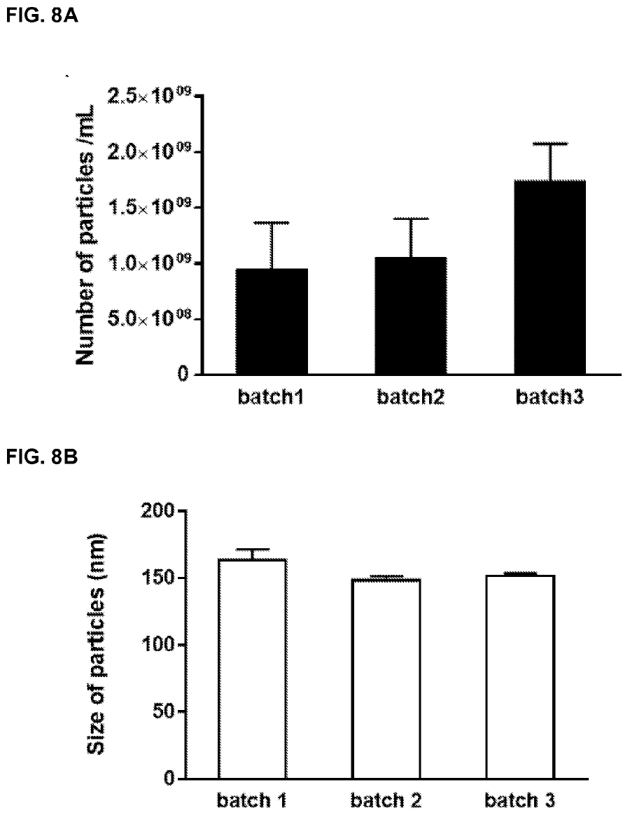

Within the mammalian CNS, EVs are known to be released by almost every type of cell, including neurons, neural stem cells, and major glial cell types such as microglia, oligodendrocytes, and astrocytes. ^{20,28-30} It has been demonstrated that astrocyte-derived EVs can be internalized in neurons but their specific physiological effect on neuron survival and function is yet to be fully understood. To address this issue, the physiological effect of astrocyte-derived EVs on neuronal survival, neurite outgrowth, and electrophysiological behavior was investigated. The homogeneity of the cultured astrocyte population was confirmed using immunostaining for two glial cell-specific protein markers, GFAP and S100B, which are known to be primarily expressed in astrocyte cytoplasm (FIGS. 2A and 2B)^{31,32}. The astrocytes were cultured for 84 hours before EV collection, and purified using serial ultracentrifugation steps with various centrifugal forces to remove non-EV elements from the culture media (FIG. 7). The collected EVs from multiple batches were characterized using nanoparticle tracking analysis, which provided size distribution and density information for all analyzed particles. The Brownian motion of the collected EVs in the assay solution was tracked by laser to correlate their speed of Brownian motion to the particle size. The size of the particles in the EV solution was 154.6±4.36 nm with a density of 1.5×109 particles/mL (FIG. 2C and FIGS. 8A and 8B).

The majority of non-EV components, such as dead cell debris, apoptotic bodies and microvesicles, are all known to have larger sizes than EVs. As such, the collected data indicated successful removal of these elements during the EV collection process.

The number of secreted EVs in the culture medium following different incubation times was investigated. The number of collected EVs were linearly proportional to the incubation time of the astrocytes, as they continuously proliferate in culture but presumably maintain relatively consistent EV secretion rates. Although there was no significant difference in the number of collected EVs between 48 and 84 hours of astrocyte culture, all cultures were maintained to 84 hours to obtain the maximum number of EVs before cells became over confluent and started to deteriorate (FIG. 2E).

### Example 3: Differentiated Cortical Neurons Readily Uptake Astrocyte-Derived EVs

EVs from glial cells can encapsulate a range of proteins and RNAs that can have a significant impact on the phenotype of the recipient neurons. ^{15,19,21,33} To deliver their cargo molecules to the recipient cell, transmembrane proteins on the membranes of both the EV and the neuron initiate their interaction to internalize the vesicle to the cell, or interact with the endosomal membrane after direct endocytic uptake. ¹⁴ Although the molecular mechanism underpinning EV uptake by mammalian cells is not fully understood, and further study is required to reveal the rules underlying glial-derived EV entry into neurons, solid evidence exists to support that EVs are well internalized by adjacent cells.^{14,33}

EV uptake can be visualized using EV-specific antibodies targeting membrane proteins rich in most EVs, or using fluorescent lipid membrane dyes to directly stain EV membranes prior to uptake. Alternatively, membrane permeable chemical compounds are also routinely used to stain EVs, which can be confined to the cytosolic lumen, emitting fluorescence as a result of esterification.^{34,35} Extracellular vesicles can be visualized in a more exclusive way via fluorescently tagged vesicular proteins that bind to the proteins expressed on the membrane of recipient cells. For example, CD81, CD9 and CD63 are tetraspanin proteins found in vesicular membranes that, when tagged with a fluorophore, can be used to visualize the uptake of vesicles into the recipient cells.³⁶

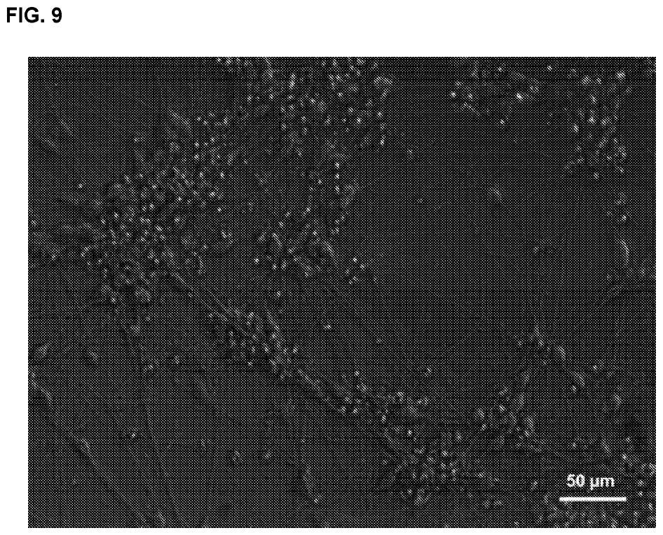

While these protein markers are used to distinguish various types of extracellular vesicles from each other, it does not offer conclusive data for distinguishing the type of vesicles observed. However, for the time being, CD81 is thought to be a relatively accurate EV-specific marker, based on its high expression on EVs but not on other type of vesicles. Conversely, CD9 and CD63 are expressed more generally throughout the majority of extracellular vesicle populations. ³⁷ Therefore, it was investigated whether astrocyte-derived EVs can be readily internalized by human cortical neurons using the ExoGlow-protein EV labeling kit and antibodies against CD81 together to cross-check EV-specific protein expression in the neuronal cytoplasm. The fluorescence signal from ExoGlow was optimized to monitor uptake of EVs by cultured neurons, but decayed rapidly after 2 hours. As such, Exoglow was only used for monitoring real-time EV uptake at the initial stages of EV endocytosis using a live cell imaging microscope. Antibodies for CD81 were then used to fluorescently quantify the internalized EVs after neuron fixation and following treatment with different ratios of cell to EV. Neurons at day 7 in culture were treated with EVs labeled by ExoGlow reagents, and the internalization rate of the EVs was then recorded. After 1 hour EV treatment and subsequent medium washing, 83.3% of neurons examined showed red fluorescence in their cell bodies in average, indicating successful uptake of astrocyte-derived EVs to the cytoplasm of cortical neurons in very short period of time (FIG. 9).

CD81 expression analysis on neurons fixed at day 7 post-induction and after 24 hours of EV treatment revealed that EV-treated neurons exhibited significant increases in fluorescence intensity in a dose-dependent manner, providing further evidence of successful internalization of labeled EVs into the cultured neurons (FIG. 2H). Fluorescence intensity dramatically increased up to the 25 EVs per cell level and then began to plateau so that higher EV concentrations did not promote further significant changes in fluorescence intensity, suggesting that EV uptake became saturated near the 1:25 ratio (FIG. 2J). The untreated batch of neurons did not show significant CD81 positivity, not only indicating the absence of EVs in the cell but also negligible levels of endogenous CD81 expression in the cultured neurons.

### Example 4: Astrocyte-Derived EV Treatment Inhibits Neuronal Apoptosis and Senescence in Cultured Neurons

The effect of astrocyte-derived EVs on cultured neuron apoptosis and senescence was studied. Since differentiation is already a very stressful process to cells in culture, the rapid differentiation caused by Neurogenin-2 overexpression could be likely even more stressful for cells, producing high rate of apoptotic neurons. 92% of neurons without EV treatment expressed at least one of the apoptotic markers used, whereas EV treated neurons showed reduced population of apoptotic cells. Increasing concentrations of EV treatments were found to correlate closely with the percentage of healthy, living cells in each group, strongly implying a role for astrocyte-derived EVs in inhibiting neurons from entering the apoptotic pathway.

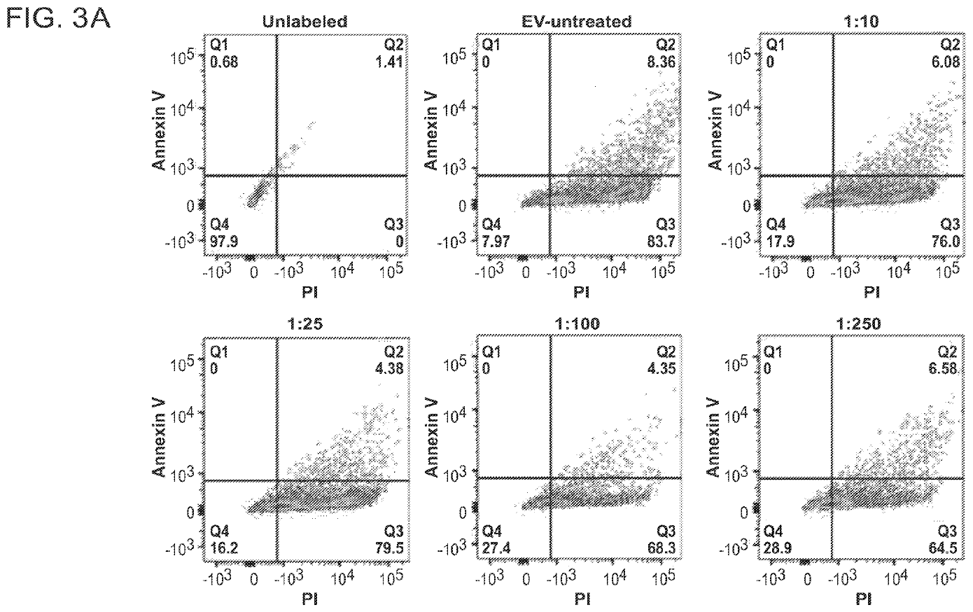

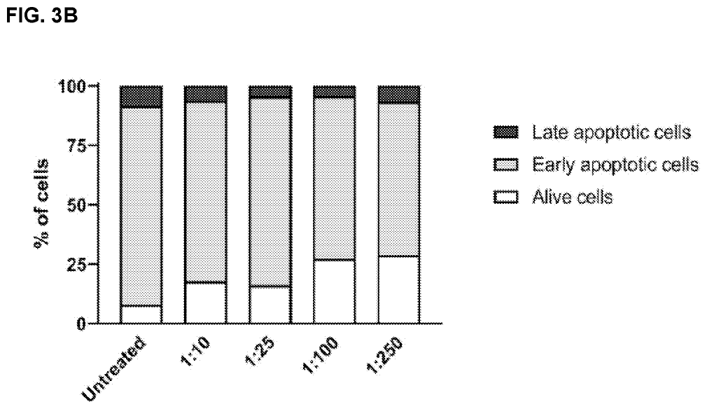

Although the EVs did not effectively rescue cells already in late stages of apoptosis, an apparent trend towards a gradual reduction in the number of cells entering early stage apoptosis in all EV-treated groups suggests that bioactive molecules encapsulated in EVs were expressed in the recipient neurons and that these signals prevented these cells from responding to pro-apoptotic signals in differentiating culture (FIGS. 3A and 3B).

In general, neuronal apoptosis represents an intrinsic suicide program by which cells initiate their own destruction as required in specific situations. ³⁸ During the development of the embryonic and neonatal nervous system, apoptosis-mediated neuronal loss contributes to regulation of the neuronal population and is responsible for removing approximately one-half of all neurons born during early neurogenesis. However, in the mature nervous system, apoptotic neuronal loss is usually physiologically inappropriate and can contribute to neurodegenerative disorders such as Alzheimer's and Parkinson's disease. ^{39,40} Though the signaling mechanisms controlling neuronal apoptosis in adult nerve tissue are not fully understood, previous studies have suggested a lack of paracrine trophic factors such as neurotrophins and IGF-1 may lead to apoptotic cell death. ³⁹ Other previous observations have led to the hypothesis that deregulation of the cell cycle can directly trigger apoptosis or oxidative stress can affect cell cycle machinery to promote apoptosis.⁴¹

The insufficient provision of growth factors and/or tolerance of oxidative stress are potent factors in driving the onset of neurodegenerative disease. As such, the lack of direct and remote support from glial cells in normal cultured neurons may lead to misinterpretation of assay results, especially in neurodegenerative disease modeling studies. It is yet to be clear which components in the astrocyte-derived EVs interact with apoptotic signaling proteins in the neurons. Nevertheless, it is clear that the astrocyte-derived EVs interact with recipient neurons in paracrine manner to improve neuronal survival and reduce apoptotic population of neurons that went through the stressful differentiation process. This may have strong implications for future in vitro studies involving such cell types.

Neural Aging

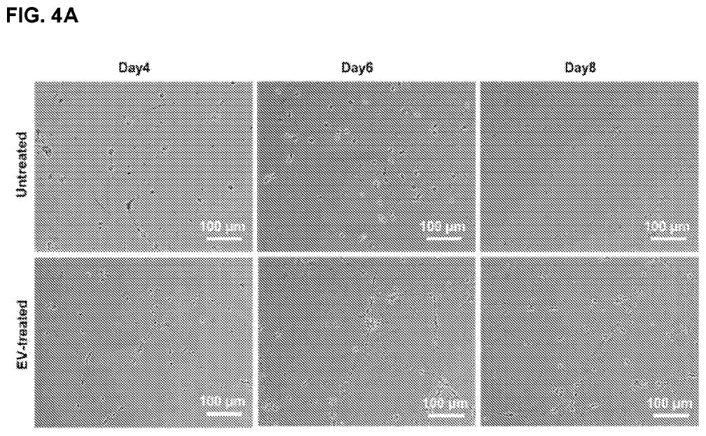

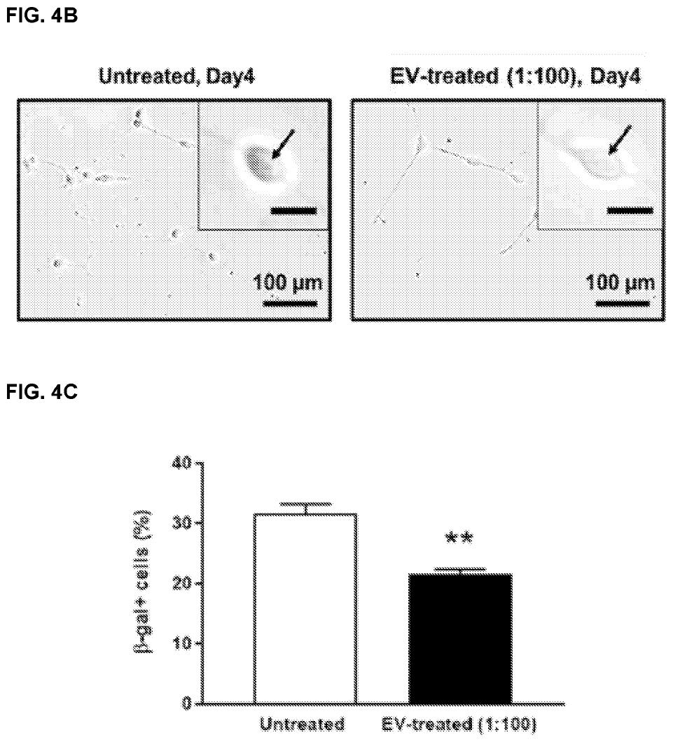

The effect of astrocyte-derived EVs on neuronal aging in low density cultures to model the loss-of-neurons observed in age-dependent neurodegenerative diseases was studied. Cortical neurons in culture can often maintain their physiology successfully on extracellular protein coated glass at high density under low-serum conditions, but low-density cultures without glial cells in vitro typically do not survive well and lack synaptogenic activities that are essential for survival and function. Early stage differentiated neurons (day 3) were plated at 10,000 cells/cm², which constitutes 10% of the typical neuron plating density for long term culture. EVs were applied 1 hour after neuron plating and medium was replenished with fresh EV-supplemented medium every three days thereafter until the assay date. Untreated groups quickly began to show significant neuritic deterioration and the cells did not survive beyond day 8 of culture. Conversely, EV-treated cultures showed normal axonal projections at day 4 and retained their robust morphology until day 8 (FIG. 4A). Additionally, it was tested whether low-density cortical neurons were susceptible to unusual cell senescence, and whether astrocyte-derived EVs inhibited their degeneration. β-galactosidase (β-gal) is a hydrolase enzyme that catalyzes the hydrolysis of βgalactosides into monosaccharides in senescent cells that normally contain hyperactive lysosomes. ⁴²

The cleavage of a chromogenic substrate X-gal solution by upregulated galactosidase activity results in the precipitation of a purple dye in the senescent cell cytoplasm. The use of β-gal as a marker of neuronal apoptosis is not without controversy as the presence of β-gal expressing neurons in fetal or young adult neurons has been interpreted alternatively as evidence that neuronal senescence is prevalent in such tissues or that β-gal is not a reliable marker for neuronal senescence. ^{43,44} Nevertheless, neuronal β-gal expression is commonly observed in stressed neural tissue.^{45 A} β-gal solution kit was used on each group of neurons and analyzed the expression of X-gal cleavage-mediated dye precipitation in their cytoplasm. Low density neurons on day 4 without EV treatment clearly expressed purple aggregates in their cell bodies, strongly indicating that the cells had adopted a senescent state (FIGS. 4B and 4C).

It is possible that neurogenin-2 transgene transduction in these cells caused the dysregulation of the neurogenin-2 protein to make them susceptible to rapid aging and degeneration. Previous studies have highlighted a correlation between dysregulation of neurogenin-2 protein and the overexpression of amyloid-β precursor protein, making this reasonable. ⁴⁶

The treatment of whether astrocyte-derived EVs suppressed the initiation of neuronal senescence was evaluated. The number of β-gal positive neurons in the EV-treated group (1:100) were significantly less than that of untreated controls at day 4 of culture, indicating that the vesicular cargo originating from astrocytes are able to inhibit neuronal senescence as well.

### Example 5: The EVs have Limited Effect on Promoting Neurite Outgrowth and Axon Branching

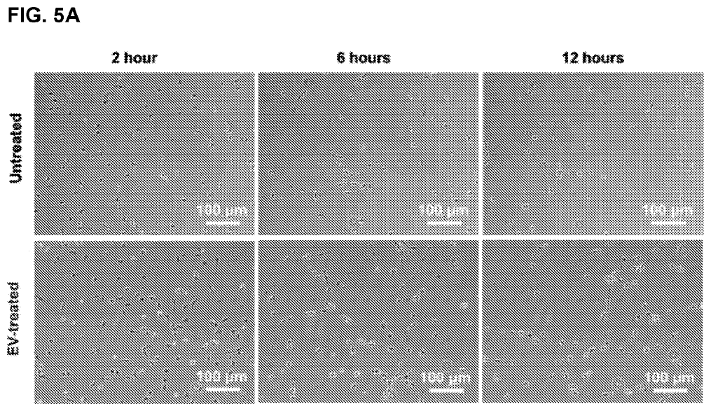

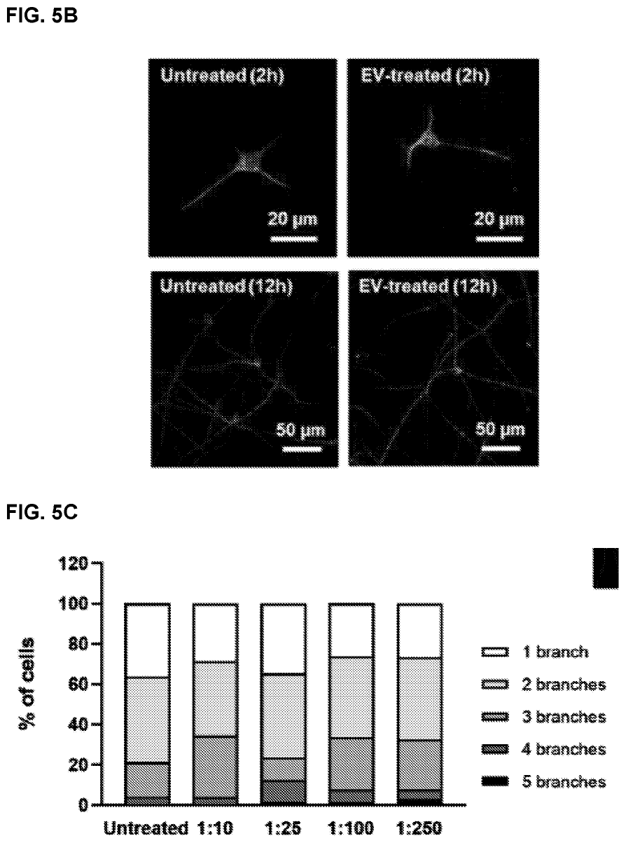

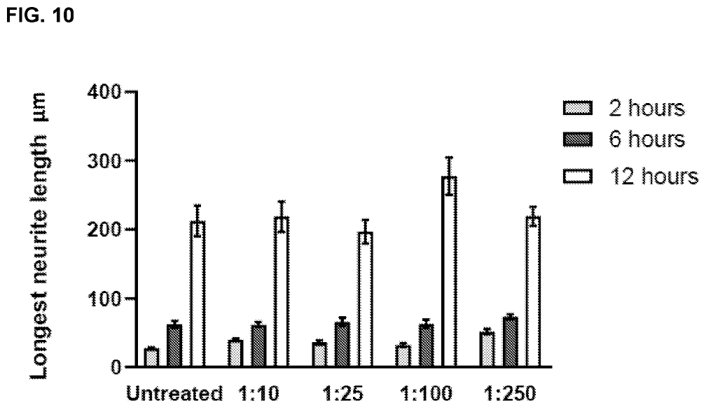

The effect of EV treatment on the kinetics of neuritic outgrowth in the early stages of culture and branching of axons at the later stages was evaluated. Neurons were treated with EVs on the day of plating and each of the cultures were imaged at different time points. The analysis of neuritic outgrowth speed showed a marked increase of the longest neurite length between 6 hours and 12 hours post-plating for every condition (FIG. 10). No significant difference in the early stage outgrowth speeds of neurites between EV untreated and EV-treated groups was observed, regardless of EV concentration examined (FIGS. 5A-C). The result indicated that the collected EVs did not contain biomolecules responsible for the regulation of neurite extension, such as proteins in the small GTPase Rho family (e.g. RhoA, Rac1, Cdc42) or miRNAs such as MiR-124, which is known to promote initial neurite growth through the control of actin polymerization.

In the early stages of neurite outgrowth, these molecules modulate the coordinated work between actin and the microtubule cytoskeleton to allow formation of the neuronal growth cone. The results presented here may indicate negligible encapsulation of those molecules in the astrocyte-derived EVs or inadequate delivery of encapsulated molecules to the recipient neurons.

Axon Branching Analysis

The effect of EVs on axon branching, defined as a process extending neurites in orthogonal angles to the main axon shaft was also evaluated. During axon development, collateral branches extend from the main axon shaft to establish diverse connections with the post-synaptic area of adjacent target cells. Gradients of molecular cues, dynamic and programmed reorganization of actin and microtubule cytoskeleton, calcium transients and intracellular signaling pathways all mediate axon branching. ⁴¹ These mechanisms are not completely understood, but it seems likely that axon growth and branching behavior into target neurons are independently regulated.

The data suggested that astrocyte-derived EVs do not significantly promote axon branching of human cortical neurons, indicating that the molecular cargos in the collected EVs do not play a role in regulating axon development (FIG. 5C).

### Example 6: Astrocyte-Derived EV Treatment Enhances Single Cell Electrophysiology of Neurons

One of the most important functions attributed to astrocytes is the buffering of extracellular potassium ions by actively taking them up from the extracellular space to prevent abnormal accumulation in the extracellular space. Proper regulation of extracellular potassium level allows neurons to maintain normal repolarization properties, assuring the proper level of neuronal excitability. ⁴⁷ Astrocytes form tripartite synapses with adjacent neurons, in which a synapse includes pre- and post-synaptic terminals between neurons, with astrocytic processes wrapped around those neuronal contacts¹⁰ Since astrocytes are actively involved in the electrochemical behavior of neurons in the CNS, and therefore may also modulate neuronal electrophysiology by means of secreted extracellular vesicles, based on the fact that the astrocytic coverage is limited to adjacent neurons, while consistent electrochemical regulation is required for neurons throughout the CNS.

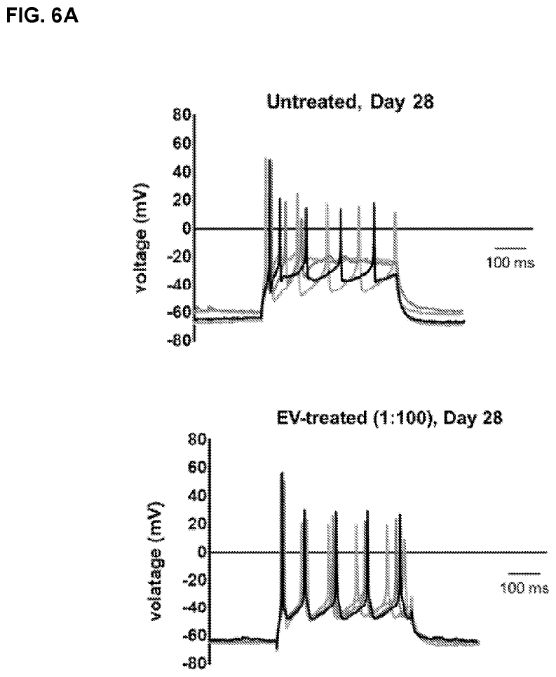

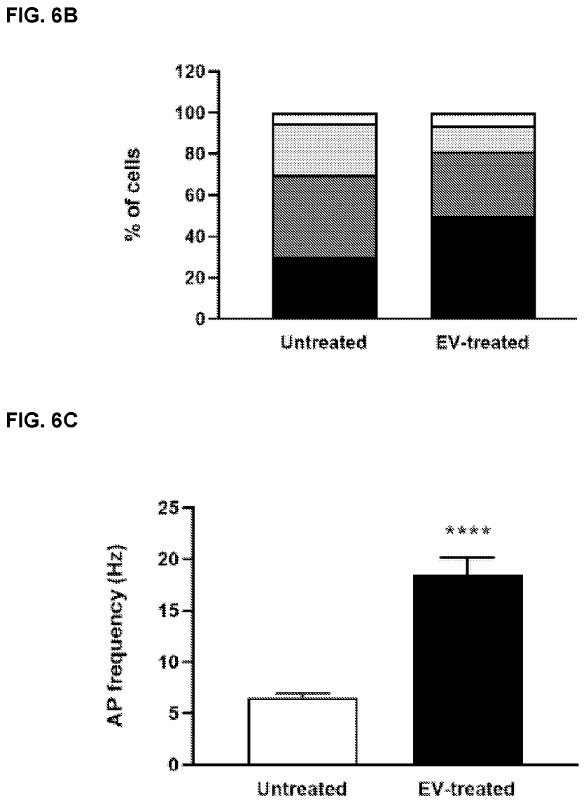

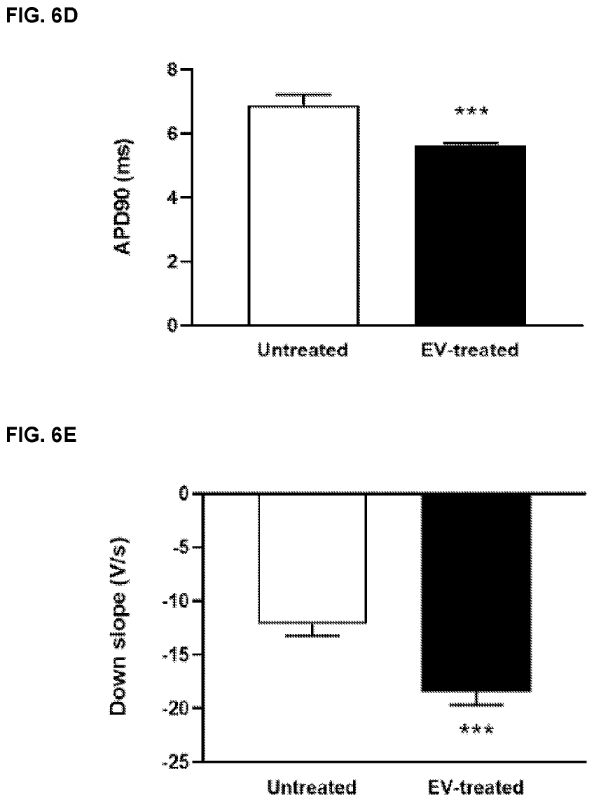

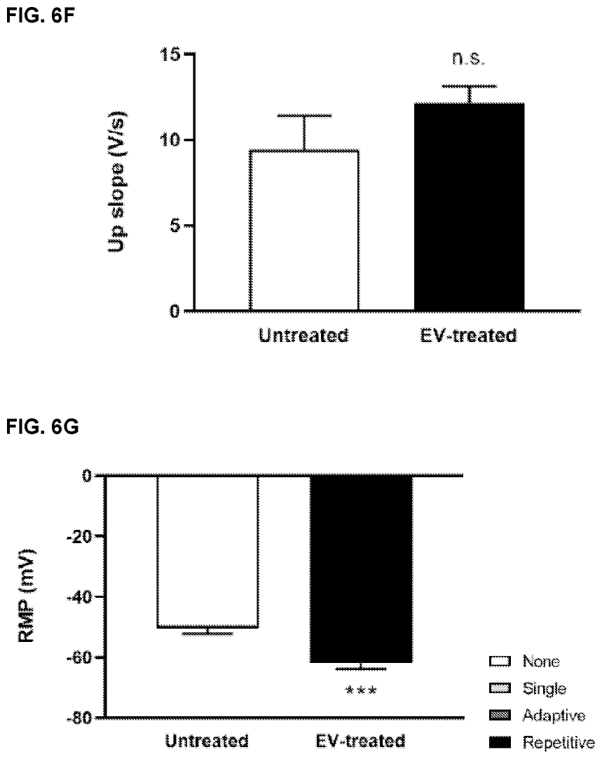

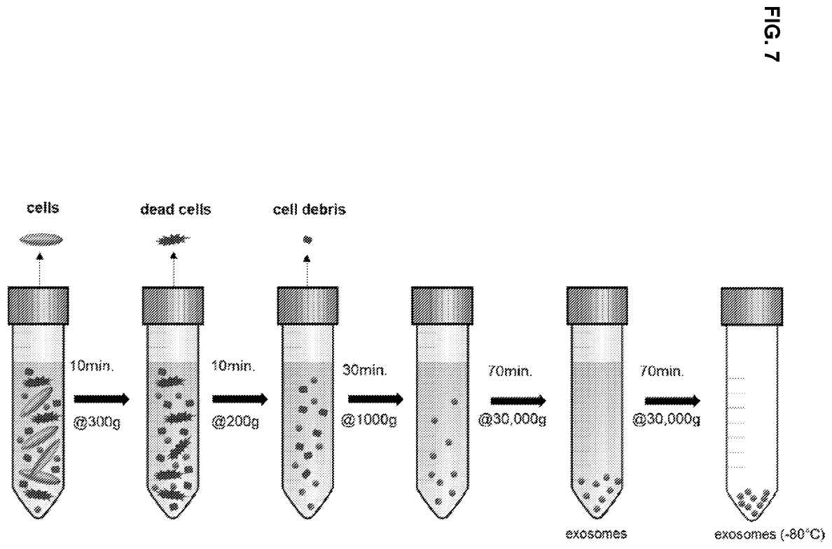

Whole cell patch-clamp recording allows for the electrophysiological assessment of single neurons, including resting membrane potential, input resistance and capacitance, and waveform analysis for evoked or spontaneous action potentials. ¹¹ The characteristics of action potentials, such as depolarization and repolarization speed, action potential amplitude and frequency, action potential duration, along with the amplitude of sodium and potassium currents, are all important criteria for the validation of functional maturation of neurons. Patch-clamp techniques were used to investigate these criteria in 4-week old cortical neuron cultures with or without treatment with astrocyte-derived EVs. Evoked action potential firing in neurons treated with EVs were more consistent, in terms of repetitive firing and action potential amplitude, than those of untreated neurons (FIG. 6A). Specifically, the population of neurons showing repetitive firing was much higher in the EV-treated group (50%) compared with that of untreated controls (30%), indicating that the EV-treated neurons were more responsive to the input stimulus than neurons cultured without the aid of astrocyte-derived EVs (FIG. 6B)

Maximum frequency of action potential firing indicates how well neurons can repeat the action potential firing cycle in response to protracted depolarizing stimuli and depends on the strength of stimulus applied to the neurons. The nervous system in the body is frequency modulated, rather than amplitude modulated, meaning that the intensity of a stimulus does not affect the action potential amplitude but the frequency of action potential firing. The maximum frequency is ultimately limited by the absolute refractory period, which is normally 1 ms in neurons in vivo. Isolated neurons in culture usually do not reach this maximum frequency of action potential firing, presumably due to the absence of glial cells and other supporting factors.

In this study, astrocyte-derived EVs clearly induced a significant increase in action potential firing frequency in cortical neurons (FIG. 6C) indicating the effect of astrocyte-mediated electrochemical modulation on neuronal function. In response to consistent current injection intensity, this differential response in action potential firing may indicate that the intracellular protein cascade that processes electrochemical stimuli in EV-assisted neurons is working more rapidly than that of untreated control cells.

Action potential duration is also a strong indicator of how rapidly neurons can cycle the influx of extracellular Na²⁺ during the depolarization period and outflux of intracellular K⁺ in the repolarization period. Shorter action potential durations at 90% of repolarization (APD90) normally indicate a swifter response of the voltage-gated ion channels on the neuron membrane and serve as an important criterion with which to evaluate cell maturity in culture. In these experiments, the astrocyte-derived EV treated cortical neurons showed significantly shorter APD90 values (5.61±0.08 ms) compared to those of the untreated group (6.89±0.33 ms) (FIG. 6D), which may indicate that the vesicular cargo molecules promoted the activity of voltage-gated ion channels to facilitate faster ion cycling. In particular, the data suggested that activity of voltage-gated potassium channels were most affected by EV treatment since a significant improvement in action potential repolarization was observed in treated cells whereas the depolarization speed was consistent (FIGS. 6E and 6F). A previous study has showed that a fast-spiking phenotype is related to the expression of the Kv3 sub-family of voltage-gated potassium channels, and the rapid voltage dependent polarization and depolarization kinetics for generating narrow action potential curves and short refractory periods are highly dependent on this channel behavior. ⁴⁸ The molecules that act to induce these changes are not yet defined, but a valuable avenue for future study would be the investigation of specific interactions between vesicular cargos and Kv3 channel development, to specify the underlying mechanism responsible for mediating this improvement.

Resting Membrane Potential (RMP) Analysis

The resting membrane potential (RMP) between treated and untreated neuronal populations was tested. The RMP is the membrane potential at the neuron's resting state, before action potential firing, and it is close to the equilibrium membrane potential of K+ (−85 mV), which is the most dominant ion in the intracellular region. The physiological value for RMP in mammalian pyramidal neurons is roughly −70 mV. Highly negative RMP values are a strong indicator of neuron maturity and indicate that cultured neuron membranes are working as an effective capacitor of ions. Cells with low RMPs maintain substantial differences in ionic concentrations between intracellular and extracellular regions, thereby facilitating the generation of a significant ionic current when an action potential is triggered. In these experiments, the average RMP of astrocyte derived EV treated neurons at day 28 in culture was −61.6±2.0 mV, whereas untreated neurons exhibited an average RMP of −50±1.76 mV (FIG. 6G). This significant decrease in RMP may indicate a greater capacity for transmembrane proteins in EV-treated cells to mediate passive K⁺ membrane permeability. Another possible speculation is that EV treatment promoted the development of a more physiological K+ gradient in the intracellular region of the cultured neurons. Such a result would therefore suggest that EV treated neurons promote the expression of more functional ion pumps and/or transporters.

The significant differences observed in critical aspects of the electrophysiology of treated and untreated groups strongly indicate a neuronal functional responsiveness to glial-derived biomolecules delivered via EVs, and the ability for these molecules to promote substantial phenotypic enhancements in relatively short culture periods.

### Example 7: Schwann Cell-Derived Exosomes

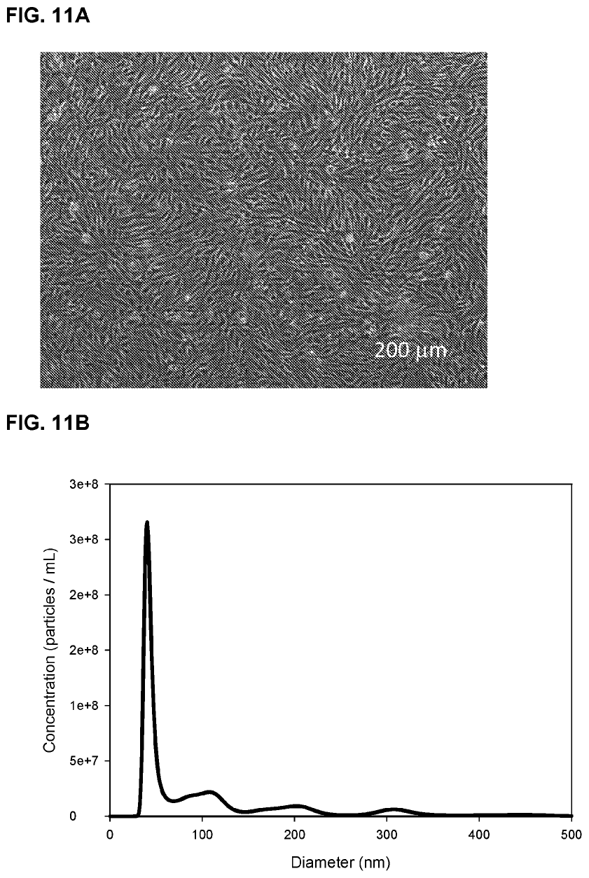

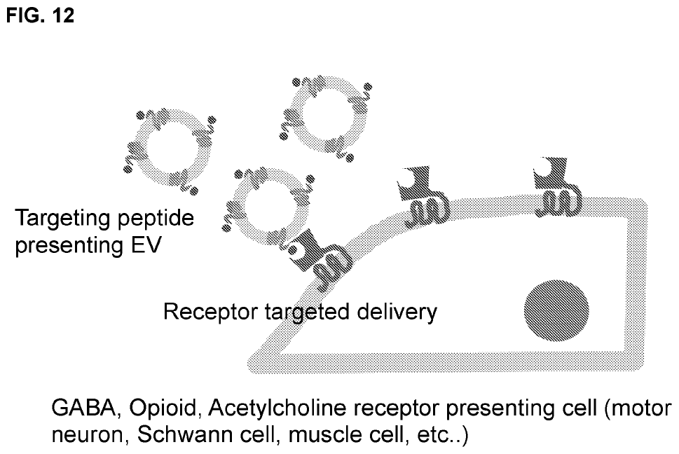

Human Schwann cell-derived exosomes were isolated and evaluated. Human Schwann cells were purchased from ScienCell Research LaboratoriesCatalog (#1700). Culture medium was purchased from ScienCell Research Laboratories (#1701). Exosome size distribution and microscopic imaging was performed with NanoSight NS300(Malvern). FIGS. 11A-11B. During the production of extracellular vesicles, cultured Schwann cells maintained their normal morphology. Moreover, it was confirmed that the extracellular vesicles generated from the Schwann cells had an average uniform size of about 100 nm.

### Example 8: Stem Cell Maturation and Scaling

Stem cell were maintained using a previously reported method^{23,24} with modifications. Specifically, the cultures were maintained with the mTeSR1 medium replenished every day for 48 more hours before being passaged and transferred to the final assay plate. Once 80% confluent stem cell colonies, the cells were dissociated into single cells using Accutase (Thermo-Fisher) and transferred on to fresh Matrigel (Corning) coated plates. Prior to EV treatment, stem cells were cultured for 24 h in growth factor supplemented neuronal maintenance medium. Then, the EV suspended medium was added to the stem cell culture with 100 EVs per cells ratio. See, e.g., Wang, C. et al. [i3N original] Scalable Production of iPSC-Derived Human Neurons to Identify Tau-Lowering Compounds by High-Content Screening. Stem Cell Reports 9, 1221-1233 (2017) and Fernandopulle, M. S. et al. [i3N protocol] Transcription Factor-Mediated Differentiation of Human iPSCs into Neurons. Curr. Protoc. Cell Biol. 79, e51 (2018), incorporated herein by reference in their entireties.

### Discussion

The extensive effects of EVs secreted from human astrocytes on human iPSC-derived cortical neurons was evaluated. Treatment with an optimized EV concentration was found to have a significant impact on inhibition of neuronal apoptosis and senescence, as well as on improving long term survival in culture. EVs also remarkably altered the electrophysiology of single neurons. A 40% increase in the number of neurons exhibiting repetitive action potential firing behavior, 3 times higher maximum frequency in action potential firing, and an 18.5% reduction in the APD90 of EV-treated neurons compared with the untreated controls was observed.

EV-treated neurons also showed a reduction in resting membrane potential and faster repolarization speed, which all indicate a positive effect of astrocyte-derived EVs on neuronal functional maturation, presumably via alterations in transmembrane protein expression and/or functionality.

The observations presented here provide significant insights on the diverse modes of neuron-glia communication in the human CNS. These data may inform on the use of EVs for developing more physiologically relevant nerve-on-a-chip assays for preclinical screening applications, enhancing the capacity to model various types of neuropathies, and even the development of novel therapies or biomarkers critical for combating neurodegenerative diseases in the future.

### Materials and Methods

The following materials and methods were used.

Astrocyte Cell Culture

Commercially sourced human primary astrocytes were purchased from Sciencell (Carlsbad, Calif.) and were stored, thawed and sub-cultured based on the manufacturer's protocol. Cultures were maintained in a 37° C./5% CO₂ incubator throughout the culture period and the astrocytes were used up to passage five, to maintain consistent cell quality. Initially, the astrocytes were cultured for 72 hours in a base medium with an astrocyte growth supplement and fetal bovine serum all provided by the same manufacturer. After 72 hours of culture, the medium was replaced with an EV-free medium consisting of the base medium plus EV-depleted serum and supplement. The cells were then cultured for an additional 84 hours, allowing them to secrete a maximal number of EVs, while maintaining less than 90% cell confluency, before collection of the EVs in the conditioned medium.

Human iPSC Maintenance and Cortical Neuron Differentiation

WTC11 human induced pluripotent stem cells stably transduced with a doxycycline-mediated Neurogenin-2 transcription factor overexpressing transgene were kindly provided by the Gladstone Institutes (San Francisco, Calif.). These cells were maintained and differentiated according to the Gladstone protocol published previously.^{23,24} The hiPSCs were differentiated into human cortical neurons using a previously reported method with limited modifications. Specifically, 80% confluent hiPSC colonies on Matrigel plates were dissociated into single cells using Accutase (Thermo-Fisher) and transferred on to fresh Matrigel (Corning) coated plates. The cells were then treated with an induction medium including N2 supplement (100×, Thermo-Fisher), non-essential amino acids (100×, Thermo-Fisher), Glutamax (100×, Thermo-Fisher) and doxycycline (1000×, Sigma-Aldrich) to initiate differentiation. The cultures were maintained with the same induction medium replenished every day for 48 more hours before being passaged and transferred to the final assay plate. Fully differentiated neurons by day 3 were transferred to 0.01% poly-L-ornithine (Sigma-Aldrich) and 5 μg/mL laminin (Sigma-Aldrich) coated glass coverslips for subsequent assays. A maintenance medium consisting of BrainPhys base medium supplemented with B27 (Invitrogen), laminin (Sigma-Aldrich), and 10 ng/mL brain derived neurotrophic factor (BDNF; R&D Systems, Minneapolis, Minn.) was fed every 3 days until endpoint analysis.

EV Isolation and Characterization

The astrocytes were cultured with vendor-provided base medium supplemented with EV-depleted serum. The culture medium in which cell-derived EVs were suspended was collected after 84 hours of culture. A multi-step ultracentrifugation protocol was then applied to concentrate and purify the EVs. First, a spin at 300 rcf for 10 minutes was used to remove accidently detached cells. 2,000 rcf for 10 minutes was then used to remove dead cells before a spin at 10,000 rcf for 30 minutes was applied to remove cell debris and apoptotic bodies. The EVs were then collected following 2 repeated ultracentrifugation steps at 30,000 rcf for 70 minutes. The EVs in the final solution were resuspended with 50 μL of BrainPhys medium (Stem cell technologies, Vancouver, BC) and stored at −80° C. for up to one month until use. The size distribution and density of the collected EVs were analyzed using a Nanoparticle Tracking Analyzer. (Nanosight of Malvern institute).

Immunocytochemistry

To verify successful differentiation of cortical neurons and homogeneity of cultured astrocytes, cells were immunostained for expression of cortical neuron (layer 2 and 3) and glia specific markers. Briefly, cells were fixed in 4% paraformaldehyde for 15 minutes and blocked with 0.5% BSA in PBS for 1 hour at room temperature. Cells were then incubated with primary antibodies diluted in 0.5% BSA solution in PBS overnight at 4° C. The next day, cells were washed 3 times with PBS. They were then incubated for 2 hours in a secondary antibody solution containing secondary antibodies diluted in 0.5% BSA solution in PBS. Coverslips were mounted on microscope slides using Vectashield containing DAPI (Vector Labs) to counterstain samples for nuclei. Images were taken at the Garvey Imaging Core at the University of Washington's Institute for Stem Cell and Regenerative Medicine using a Nikon A1 Confocal System on a Ti-E inverted microscope platform. Antibodies used in these studies were as follows: rabbit anti-MAP-2 (1:1000, Millipore), mouse anti-CUX-1 (1:500, Thermo-Fisher), chicken anti-GFAP (1:1000, Invitrogen), mouse anti-S100B (1:500, Sigma-Aldrich), Alexafluor-594 conjugated goat-anti-mouse secondary antibody (1:200, Invitrogen), Alexafluor-594 conjugated goat-anti-chicken secondary antibody (1:200, Invitrogen) Alexafluor-488 conjugated goat-anti-mouse secondary antibody (1:200, Invitrogen) and Alexafluor-647 conjugated goat-anti-rabbit secondary antibody (1:200, Invitrogen).

EV Labeling and Uptake Study

An ExoGlow-protein EV labeling kit (System Biosciences, Palo Alto, Calif.) was used for live cell EV imaging. Collected EVs were incubated for 20 minutes with the labeling dye (1:500) at 37° C. to induce vesicular protein conjugation with the dye molecules. Then the solution was treated with the ExoQuick-TC solution and incubated 2 hours at 4° C., followed by centrifugation at 10,000 rcf for 10 minutes to remove unlabeled reagent molecules and collect labeled EVs only. The labeled EVs were resuspended neuronal maintenance medium and added to neurons 72 hours post differentiation at various concentrations. A live cell imaging microscope (Eclipse T1, Nikon) was used to image fluorescently labeled EV uptake by the cultured neurons, by acquiring images at multiple locations every 5 minutes for 2 hours. Additionally, neurons treated with EVs were fixed after 2 hours of treatment and immunostained with a mouse anti-CD81 antibody (1:100, Thermo-Fisher) followed by Alexafluor-594 conjugated goat-anti-mouse secondary antibody (1:200, Invitrogen) with fluorescently-labelled phalloidin (for F-actin visualization; 1:200, Invitrogen) to quantify the relative amount of internalized EVs based on fluorescence intensity.

Cell Senescence and Apoptosis Assay

To quantify the population of senescent neurons in each culture condition, a β-galactosidase staining kit (Sigma-Aldrich) was used. The cells were fixed at day 4 with 4% PFA for 15 minutes. The staining solution was prepared by mixing 25 μL of the X-gal solution with 475 μL of iron buffer to make a total of 500 μL for each well. The staining solution was added to the culture and kept at 37° C. for 3 hours before assessment. The number of cells exhibiting blue dye accumulation in their cytoplasm under bright field microscope was then compared for each group. To quantify apoptotic cells in these cultures, Annexin 5 (Thermo-Fisher) with propidium iodide (Thermo-Fisher) was used to label cells for flow cytometry analysis (BD Canto II). Specifically, 200,000 cells per condition were treated with fluorescently labelled Annexin V and propidium iodide for 15 minutes at room temperature. These cells were then immediately washed before FACS analysis to minimize the cell death during the assay process. The number of cells expressing Annexin V and/or propidium iodide in each group was then analyzed and quantified using FloJo software (BD Biosciences).

Morphological Assessment of Neurons

To examine the effect of astrocyte-derived EV treatment on axon outgrowth, cultured neurons were imaged 2 hours, 6 hours and 12 hours after plating. The length of the longest neurite segment for each neuron was measured for every image taken at each time point. Additionally, neurons from each group were fixed at each timepoint and stained with anti-neurofilament to enable comparative assessment of axon length. The axon morphologies were analyzed using the Simple Neurite Tracer plugin in ImageJ. To measure axon branching, the neurons immunostained for neurofilament expression were individually analyzed by eye for the divergence of axon terminals.

Electrophysiological Assessment of Neurons

Electrophysiological function of neurons in each group was recorded using whole-cell patch clamp techniques. Recordings were performed on an inverted DIC microscope (Nikon) connected to an EPC10 patch clamp amplifier and computer running Patchmaster software (HEKA). Coverslips supporting cultured neurons were loaded onto the microscope stage and bathed in a Tyrode's solution containing 140 mM NaCl, 5.4 mM KCl, 1.8 mM CaCl2, 1 mM MgCl₂, 10 mM glucose, and 10 mM HEPES. The intracellular recording solution (120 mM L-aspartic acid, 20 mM KCl, 5 mM NaCl, 1 mM MgCl₂, 3 mM Mg²⁺-ATP, 5 mM EGTA, and 10 mM HEPES) was loaded into borosilicate glass patch pipettes (World Precision Instruments) with a resistance in the range of 2-6 MΩ. Offset potentials were nulled before formation of a gigaΩ seal. Suction was then applied to disrupt the cell membrane in contact with pipette end. This process allowed electrical and molecular access to the intracellular space, and membrane potentials were then corrected by subtraction of the liquid junction potential, calculated by Patchmaster. The current clamp mode was used for recording action potential behavior of neurons. Specifically, a 2 nA depolarizing single pulse was applied for 5 ms to induce single action potentials, and stepwise current injections of 10 pA from −30 to +70 pA for 500 ms were applied to trigger repetitive action potential firing. The action potential duration at 90% repolarization (APD90), depolarization speed and repolarization speed were recorded for each group of neurons by the Patchmaster software.

Inward and outward currents were evoked in voltage clamp mode, by providing 500 ms depolarizing steps from −120 mV to +30 mV in 10 mV increments. To measure resting membrane potential of neurons in each group, gap-free recordings of spontaneous activity in patched neurons were performed in current-clamp mode with 0 pA current injection. The analyses of action potential waveforms and currents were performed using the Patchmaster software suite. All reagents used in this protocol were obtained from Sigma-Aldrich.

### REFERENCES

1. Farhy-Tselnicker, I. & Allen, N. J. Astrocytes, neurons, synapses: A tripartite view on cortical circuit development. Neural Dev. 13, 1-12 (2018).
2. Molnar, Z. et al. New insights into the development of the human cerebral cortex. J. Anat. 432-451 (2019). doi:10.1111/joa.13055
3. Rakic, P. & Lombroso, P. J. Development of the cerebral cortex: I. Forming the cortical structure. J. Am. Acad. Child Adolesc. Psychiatry 37, 116-117 (1998).
4. Harris, K. D. & Mrsic-Flogel, T. D. Cortical connectivity and sensory coding. Nature 503, 51-58 (2013).
5. Douglas, R. J. & Martin, K. A. C. Neuronal Circuits of the Neocortex. Annu. Rev. Neurosci. 27, 419-451 (2004).
6. Dzyubenko, E., Gottschling, C. & Faissner, A. Neuron-Glia Interactions in Neural Plasticity: Contributions of Neural Extracellular Matrix and Perineuronal Nets. Neural Plast. 2016, (2016).
7. Han, X. et al. NIH Public Access. 12, 342-353 (2014).
8. Luarte, A. et al. Astrocytes at the Hub of the Stress Response: Potential Modulation of Neurogenesis by miRNAs in Astrocyte-Derived Exosomes. Stem Cells Int. 2017, (2017).
9. Shi, Y., Kirwan, P. & Livesey, F. J. Directed differentiation of human pluripotent stem cells to cerebral cortex neurons and neural networks. Nat. Protoc. 7, 1836-1846 (2012).
10. Bellot-Saez, A., Kékesi, O., Morley, J. W. & Buskila, Y. Astrocytic modulation of neuronal excitability through K+spatial buffering. Neurosci. Biobehav. Rev. 77, 87-97 (2017).
11. Hansen, M. G., Tornero, D., Canals, I., Ahlenius, H. & Kokaia, Z. In vitro functional characterization of human neurons and astrocytes using calcium imaging and electrophysiology. Methods Mol. Biol. 1919, 73-88 (2019).
12. Johnstone, R. M., Adam, M., Hammond, J. R., Orr, L. & Turbide, C. Vesicle formation during reticulocyte maturation. Association of plasma membrane activities with released vesicles (exosomes). J. Biol. Chem. 262, 9412-9420 (1987).
13. Lee, Y., El Andaloussi, S. & Wood, M. J. A. Exosomes and microvesicles: Extracellular vesicles for genetic information transfer and gene therapy. Hum. Mol. Genet. 21, 125-134 (2012).
14. Mulcahy, L. A., Pink, R. C., Raul, D. & Carter, F. Routes and mechanisms of extracellular vesicle uptake. 1, 1-14 (2014).
15. Saeedi, S., Israel, S., Nagy, C. & Turecki, G. The emerging role of exosomes in mental disorders. Transl. Psychiatry 9, (2019).
16. Gangoda, L., Boukouris, S., Liem, M., Kalra, H. & Mathivanan, S. Extracellular vesicles including exosomes are mediators of signal transduction: Are they protective or pathogenic? Proteomics 15, 260-271 (2015).
17. Zagrean, A. M., Hermann, D. M., Opris, I., Zagrean, L. & Popa-Wagner, A. Multicellular crosstalk between exosomes and the neurovascular unit after cerebral ischemia. therapeutic implications. Front. Neurosci. 12, 1-9 (2018).
18. Paolicelli, R. C., Bergamini, G. & Rajendran, L. Cell-to-cell Communication by Extracellular Vesicles: Focus on Microglia. Neuroscience 405, 148-157 (2019).
19. Basso, M. & Bonetto, V. Extracellular vesicles and a novel form of communication in the brain. Front. Neurosci. 10, 1-13 (2016).
20. Goetzl, E. J. et al. High complement levels in astrocyte-derived exosomes of Alzheimer's disease. 83, 544-552 (2019).
21. Pascua-Maestro, R. et al. Extracellular vesicles secreted by astroglial cells transport apolipoprotein D to neurons and mediate neuronal survival upon oxidative stress. Front. Cell. Neurosci. 12, 1-13 (2019).
22. Mizuno, G. O. et al. Aberrant Calcium Signaling in Astrocytes Inhibits Neuronal Excitability in a Human Down Syndrome Stem Cell Model. Cell Rep. 24, 355-365 (2018).
23. Wang, C. et al. [i3N original] Scalable Production of iPSC-Derived Human Neurons to Identify Tau-Lowering Compounds by High-Content Screening. Stem Cell Reports 9, 1221-1233 (2017).
24. Fernandopulle, M. S. et al. [i3N protocol] Transcription Factor-Mediated Differentiation of Human iPSCs into Neurons. Curr. Protoc. Cell Biol. 79, e51 (2018).
25. Shi, Y., Kirwan, P. & Livesey, F. J. Directed differentiation of human pluripotent stem cells to cerebral cortex neurons and neural networks. Nat. Protoc. 7, 1836-1846 (2012).
26. Patani, R. et al. Retinoid-independent motor neurogenesis from human embryonic stem cells reveals a medial columnar ground state. Nat. Commun. 2, (2011).
27. Dien et al., 2013. [symbol] NIH Public Access. Bone 23, 1-7 (2008).
28. Polanco, J. C., Li, C., Durisic, N., Sullivan, R. & Götz, J. Exosomes taken up by neurons hijack the endosomal pathway to spread to interconnected neurons. Acta Neuropathol. Commun. 6, 10 (2018).
29. Janas, A. M., Sapoń, K., Janas, T., Stowell, M. H. B. & Janas, T. [2016 Review] Exosomes and other extracellular vesicles in neural cells and neurodegenerative diseases. Biochim. Biophys. Acta—Biomembr. 1858, 1139-1151 (2016).
30. Sharma, P., Mesci, P., Carromeu, C., Mcclatchy, D. R. & Schiapparelli, L. Exosomes regulate neurogenesis and circuit assembly. 1-9 (2019). doi:10.1073/pnas.1902513116
31. Harrison, J. F. et al. Notch Signaling Modulates the Activation of Microglial Cells. Glia 55, 1416-1425 (2007).
32. Yasuda, Y. et al. Relationship between S100β and GFAP expression in astrocytes during infarction and glial scar formation after mild transient ischemia. Brain Res. 1021, 20-31 (2004).
33. Frühbeis, C., Fröhlich, D. & Kramer-Albers, E. M. Emerging roles of exosomes in neuronglia communication. Front. Physiol. 3 April, 1-7 (2012).
34. Hessvik, N. P. & Llorente, A. Current knowledge on exosome biogenesis and release. Cell. Mol. Life Sci. 75, 193-208 (2018).
35. Skotland, T., Hessvik, N. P., Sandvig, K. & Llorente, A. Exosomal lipid composition and the role of ether lipids and phosphoinositides in exosome biology. J. Lipid Res. 60, 9-18 (2019).
36. Andreu, Z. & Yáñez-Mó, M. Tetraspanins in extracellular vesicle formation and function. Front. Immunol. 5, 1-12 (2014).
37. Kowal, J. et al. Proteomic comparison defines novel markers to characterize heterogeneous populations of extracellular vesicle subtypes. Proc. Natl. Acad. Sci. U.S.A. 113, E968-E977 (2016).
38. Cavallaro, S. Cracking the code of neuronal apoptosis and survival. Cell Death Dis. 6, e1963-3 (2015).
39. Hussain, R., Zubair, H., Pursell, S. & Shahab, M. Neurodegenerative diseases: Regenerative mechanisms and novel therapeutic approaches. Brain Sci. 8, (2018).
40. Spalding, K. L. et al.)(Dynamics of hippocampal neurogenesis in adult humans. Cell 153, 1219 (2013).
41. Klein, J. A. & Ackerman, S. L. Oxidative stress, cell cycle, and neurodegeneration. J. Clin. Invest. 111, 785-793 (2003).
42. Lee, B. Y. et al. Senescence-associated β-galactosidase is lysosomal β-galactosidase. Aging Cell 5, 187-195 (2006).
43. Piechota, M. et al. Is senescence-associated β-galactosidase a marker of neuronal senescence? Oncotarget 7, 81099-81109 (2016).
44. de Mera-Rodriguez, J. A. et al. Senescence-associated β-galactosidase activity in the developing avian retina. Dev. Dyn. 248, 850-865 (2019).
45. Tominaga, T., Shimada, R., Okada, Y., Kawamata, T. & Kibayashi, K. Senescence associated-β-galactosidase staining following traumatic brain injury in the mouse cerebrum. PLoS One 14, 1-17 (2019).
46. Bolós, M., Hu, Y., Young, K. M., Foa, L. & Small, D. H. Neurogenin 2 mediates amyloid-β precursor protein-stimulated neurogenesis. J. Biol. Chem. 289, 31253-31261 (2014).
47. Bordey, A., Lyons, S. A., Hablitz, J. J. & Sontheimer, H. Electrophysiological characteristics of reactive astrocytes in experimental cortical dysplasia. J. Neurophysiol. 85, 1719-1731 (2001).
48. Labro, A. J., Priest, M. F., Lacroix, J. J., Snyders, D. J. & Bezanilla, F. Kv 3.1 uses a timely resurgent K+current to secure action potential repolarization. Nat. Commun. 6, 1-12 (2015).
49. Devlin, A. et al. Human iPSC-derived motoneurons harbouring TARDBP or C9ORF72 ALS mutations are dysfunctional despite maintaining viability. Nat. Commun. 6, 1-12

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All references, e.g., U.S. patents, U.S. patent application publications, pct patent applications designating the U.S. published foreign patents and patent applications cited herein are incorporated herein by reference in their entireties. Genbank and ncbi submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

While this invention has been particularly shown and described with references to preferred embodiments thereof, it will be understood by those skilled in the art that various changes in form and details may be made therein without departing from the scope of the invention encompassed by the appended claims.

1. A composition comprising a glial-derived extracellular vesicle, and wherein the extracellular vesicle comprises one or more of the following miRNA, an adeno-associated virus (AAV), siRNA, vRNA, mRNA, lncRNA, DNA, tetraspanins, amino acids, metabolites, signaling proteins, chaperones, cytoskeletal proteins, enzymes, or combinations thereof.

2. The composition of claim 1, wherein the glial-derived extracellular vesicle comprises miRNA-21, miRNA-132, miRNA-9, miRNA-27a, miRNA-221, miRNA-200a-3p, miRNA-361, miRNA-274, miR-873a-5p, AAV1, AAV2, AAV4, AAV5, AAV6, AAV7, AAV8, AAV9 AAVDJ8, AAVrh10, glial derived neurotrophic factor, brain derived neurotrophic factor, Ciliary neurotrophic factor, GFAP, or combinations thereof.

3. The composition of claim 1, wherein the glial-derived extracellular vesicle are derived from astrocytes, Schwann cells, oligodendrocytes, ependymal cells, microglia, or satellite cells in 2D or 3D cultures.

4. The composition of claim 3, wherein the cells are mammalian primary isolated cells or mammalian stem cells.

5. The composition of claim 4, wherein the stem cells comprise induced pluripotent stem cells, embryonic stem cells, or mesenchymal stem cells.

6. The composition of claim 1, wherein the extracellular vesicle comprises AAV9, AAVDJ8, AAVrh10, AAV6, AAV5, AAV1, or AAV2.

7. The composition of claim 1, wherein the glial-derived extracellular vesicle comprises a genetically modified protein or fragment thereof for expressing the protein or fragment thereof on the surface of the extracellular vesicle.

8. The composition of claim 7, wherein the genetically modified protein comprises Lamp-1, Lamp-2, tetraspanins, CD2, CD3, CD9, CD13, CD18, CD36, CD37, CD40, CD40L, CD41a, CD44, CD45, CD53, CD63, CD81, CD82, CD86, Flotillin, Syntaxin-3, ICAM-1, Integrin alpha4, LiCAM, LFA-1, Mac-1, Vti-1A and B, CXCR4, FcR, GluR2/3, HLA-DM, Immunoglobulins, MHC-1, MHC-2, or TCR beta.

9. The composition of claim 7, wherein the genetically modified protein comprises at least one of a rabies virus glycoprotein (RVG), a tetanus toxin fragment C, or an RGD peptide.

10. The composition of claim 1, wherein the extracellular vesicle has a diameter from about 10 nm to about 1000 nm.

11. (canceled)

12. A method for preparing the extracellular vesicle of claim 1, comprising:
culturing cells in a medium, wherein the cells release the extracellular vesicle by secretion into the medium,
collecting the supernatant of medium, fractionating the supernatant comprising the extracellular vesicle, and isolating the extracellular vesicle.

13-16. (canceled)

17. A method of treating a neuropathy in a subject comprising administering to the subject an effective amount of a composition of claim 1.

18. The method of claim 17, wherein the neuropathy comprises traumatic peripheral nerve injury, chemotherapy induced peripheral neuropathy, traumatic brain injury, stroke, Charcot-Marie-Tooth disease (CMT), Amyotrophic Lateral Sclerosis (ALS), Parkinson's disease, Alzheimer's disease, frontotemporal dementia, Huntington's disease, Multiple Sclerosis, Congenital Myasthenia, Apraxia, Hypertonia, myasthenia gravis, or spinal muscular atrophy.

19-24. (canceled)

25. A method for preventing or treating neuronal apoptosis, neuronal senescence, neuritic outgrowth, synapse function or electrophysiological function in a subject, comprising administering to the subject an effective amount the composition of claim 1.

26-30. (canceled)

31. The method of claim 25 further comprising:
identifying a subject as suffering from neuronal apoptosis, neuronal senescence, neuritic outgrowth, synapse function or electrophysiological function; and
administering the composition to the identified subject.

32. The method of claim 25 further comprising:
identifying a subject as susceptible to neuronal apoptosis, neuronal senescence, neuritic outgrowth, synapse function or electrophysiological function; and
administering the composition to the identified subject.

33. The method of claim 25, wherein the subject is a human.

34. A composition comprising a glial-derived extracellular vesicle, and wherein the extracellular vesicle comprises one or more gene editing tools, wherein the gene editing tools include a gene editing protein, an RNA molecule and/or a ribonucleoprotein.

35-37. (canceled)

38. A method for treating a population of cells in vitro, comprising: administering to the cells in vitro a composition of claim 1.

39. (canceled)

40. A population of stem cells or progenitor cells produced by the method of claim 38.
